(19) United States
(12) Patent Application Publication
(10) Pub. No.: US 2010/0178353 A1
(43) Pub. Date: Jul. 15, 2010

(54) QUICK DISSOLVE COMPOSITIONS AND TABLETS BASED THEREON

(75) Inventors: Naima MEZAACHE, McLean, VA (US); Steven E. Frisbee, Reston, VA (US); Patrick B. Woodall, Culpeper, VA (US); Mark R. Herman, Nokesville, VA (US)

Correspondence Address: OBLON, SPIVAK, MCCLELLAND MAIER & NEUSTADT, L.L.P., 1940 DUKE STREET, ALEXANDRIA, VA, 22314, US

(73) Assignee: BIOVAIL LABORATORIES INTERNATIONAL S.R.L., St. Michael (BB)

(21) Appl. No.: 12/730,339

(22) Filed: Mar. 24, 2010

Related U.S. Application Data
(63) Continuation of application No. 10/176,135, filed on Jun. 21, 2002.
Application No. 10/176,135 is a continuation-in-part of application No. 09/179,926, filed on Oct. 27, 1998, now abandoned.

(51) Int. Cl. A61K 9/14; A61K 31/138; A61K 31/4525; A61K 31/437; A61P 25/24

(52) U.S. Cl. 424/494; 424/497; 514/651; 514/321; 514/300; 424/490

(57) ABSTRACT

The invention provides a composition useful for making oral dosage forms capable of dissolving in the mouth in less than 40 seconds without the need for a conventional super disintegrant and having a friability of less than 1%; wherein the composition includes liquiflash particles and an excipient mass. A preferred excipient mass according to the invention contains a directly compressible inorganic salt; a cellulose derivative or a combination of a directly compressible inorganic salt and a cellulose derivative. Preferably, the liquiflash particles and the excipient mass are combined in proportions such that the active ingredient remains substantially within the microspheres when the composition is compressed to obtain a dosage form having a hardness of 20 to 50 N. The compositions of the invention allow for the fabrication of oral dosages having improved hardness and friability.

### RELATED APPLICATIONS

This application is a Continuation application of U.S. application Ser. No. 10/176,135, filed Jun. 21, 2002, now allowed, which is a Continuation-in-Part application of U.S. application Ser. No. 09/179,926 filed Oct. 27, 1998, the content of which are hereby incorporated by reference in their entirety.

### FIELD OF THE INVENTION

The invention relates to compressible compositions and dosage forms based thereon, such as tablets and lozenges, which, when ingested, quickly dissolve in the mouth, but which effectively mask the taste of unpleasant active agent(s) therein. Also, the invention relates to readily processable compositions having enhanced friability and hardness properties which permit shaping, e.g., tableting, without the need for complex packaging equipment.

### BACKGROUND

The post-genomics phase in the life sciences arena has brought an increased yield of new small molecules that are pursued to target particular diseases based on the new understanding of the molecular basis of disease. The tremendous progress achieved in molecular structural biology has allowed the identification and de novo design of efficient molecules or so called “smart drugs.” The new technologies based on the unraveling of the human genome, the intensive progress in elucidating the structures of the enzymes encoded therein combined with the efficiencies of combinatorial chemistry will continue to generate small molecules that need to be administered to patients in efficient and organoliptically acceptable forms. One aspect associated with ameliorating the effects of ingesting molecules that are generally unpalatable is to provide the drug in dosage forms, such as tablets and lozenges, which, when ingested, quickly dissolve in the mouth.

Tablets may be defined as solid dosage pharmaceutical forms containing drug substances with or without suitable fillers. They are produced by compression or compaction of a formulation containing the drug and certain excipients selected to aid in the processing and to improve the properties of the product. Tablets may be coated or uncoated and are made from powdered, crystalline materials. They may include various diluents, binders, disintegrants, lubricants, glidants and in many cases, colorants. Excipients used are classified according to the function they perform. For example, a glidant may be used to improve the flow of powder blend in the hopper and into the tablet die.

There has been widespread use of tablets since the latter part of the 19th century and the majority of pharmaceutical dosage forms are marketed as tablets. Major reasons of tablet popularity as a dosage form among pharmaceutical manufacturers are simplicity, low cost, and the speed of production. Other reasons include stability of drug product, convenience in packaging, shipping, and dispensing. To the patient or consumer, tablets offer convenience of administration, ease of accurate dosage, compactness, portability, blandness of taste, ease of administration, and elegant distinctive appearance.

Tablets may be plain, film or sugar coated, bisected, embossed, layered, or sustained release. They can be made in a variety of sizes, shapes and colors. Tablets may be swallowed, chewed, or dissolved in the buccal cavity or beneath the tongue. They may be dissolved in water for local or topical application. Sterile tablets are normally used for parenteral solutions and for implantation beneath the skin.

In addition to the active or therapeutic ingredients, tablets may contain a number of inert materials known as excipients. They may be classified according to the role they play in the final tablet. The primary composition includes a filler, binder, lubricant, and glidant. Other excipients which give physical characteristics to the finished tablet are coloring agents, and flavors in the case of chewable tablets. Without excipients most drugs and pharmaceutical ingredients cannot be directly compressed into tablets. This is primarily due to the poor flow and cohesive properties of most drugs. Typically, excipients are added to a formulation to impart good flow and compression characteristics to the material being compressed. Such properties are imparted to these excipients through pretreatment steps such as wet granulation, slugging, spray drying spheronization, or crystallization.

Lubricants are typically added to prevent the tableting materials from sticking to punches, minimize friction during tablet compression, and allow for removal of the compressed tablet from the die. Such lubricants are commonly included in the final tablet mix in amounts usually less than 1% by weight.

In addition, tablets often contain diluents which are added to increase the bulk weight of the blend resulting in a practical size for compression. This is often necessary where the dose of the drug is relatively small.

Another commonly used class of excipients in tablets is binders. Binders are agents, which impart cohesive qualities to the powdered material. Commonly used binders include starch, and sugars such as sucrose, glucose, dextrose, and lactose.

Disintegrants are often included to ensure that the tablet has an acceptable rate of disintegration. Typical disintegrants include starch derivatives and salts of carboxymethylcellulose.

Other desirable characteristics of excipients include the following:
High compressibility to allow strong tablets to be made at low compression forces.
Good flow properties that can improve the flow of other excipients in the formula.
Cohesiveness (to prevent tablet from crumbling during processing, shipping and handling).

The three processes for making compressed tablets are wet granulation, direct compression, and dry granulation (slugging or roller compaction). The method of preparation and type of excipients are selected to give the tablet formulation the desired physical characteristics that allow for the rapid compression of the tablets. After compression, the tablets must have a number of additional attributes such as appearance, hardness, disintegrating ability, and an acceptable dissolution profile. Choice of fillers and other excipients will depend on the chemical and physical properties of the drug, behavior of the mixture during processing, and the properties of the final tablets. Preformulation studies are done to determine the chemical and physical compatibility of the active component with proposed excipients.

The properties of the drug, its dosage forms, and the economics of the operation will determine selection of the best process for tableting. Generally, both wet granulation and direct compression are used in developing a tablet.

The dry granulation method may be used where one of the constituents, either the drug or the diluent, has sufficient cohesive properties to be tableted. The method consists of blending, slugging the ingredients, dry screening, lubrication, and compression.

The wet granulation method is used to convert a powder mixture into granules having suitable flow and cohesive properties for tableting. The procedure consists of mixing the powders in a suitable blender followed by adding the granulating solution under shear to the mixed powders to obtain a granulation. The damp mass is then screened through a suitable screen and dried by tray drying or fluidized bed drying. Alternately, the wet mass may be dried and passed through a mill. The overall process includes: weighing, dry powder blending, wet granulating, drying, milling, blending lubrication and compression.

In general, powders do not have sufficient adhesive or cohesive properties to form hard, strong granules. A binder is usually required to bond the powder particles together due to the poor cohesive properties of most powders. Heat and moisture sensitive drugs cannot usually be manufactured using wet granulation. The large number of processing steps and processing time are problems due to high level manufacturing costs. Wet granulation has also been known to reduce the compressibility of some pharmaceutical excipients such as microcrystalline cellulose.

Direct compression is regarded as a relatively quick process where the powdered materials are compressed directly without changing the physical and chemical properties of the drug. The active ingredient(s), direct compression excipients and other auxiliary substances, such as a glidant and lubricant are blended in a twin shell blender or similar low shear apparatus before being compressed into tablets. This type of mixing was believed to be essential in order to prepare “pharmaceutically acceptable” dosage forms. For example, Remington's Pharmaceutical Sciences (RPS), pp 1203 to 1932 17.sup.th edition (1985), cautions pharmaceutical scientists that the manner in which a lubricant is added to a formulation must be carefully controlled.

Accordingly, lubricants are usually added to a granulation by gentle mixing. RPS warns that prolonged blending of a lubricant with a granulation can materially affect hardness and disintegration time for the resulting tablets. Furthermore, Ansel et al (1995) Pharmaceutical Dosage Forms and Drug Delivery Systems, 6.sup.th Ed. p. 199, indicates that excessive blending of lubricants with the granulate ingredients cause water proofing of the granule and reduces tablet hardness or strength of the compressed tablet. For these reasons, high shear mixing conditions have not been used to prepare direct compression dosage forms.

The advantages of direct compression include uniformity of blend, few manufacturing steps involved, (i.e. the overall process involves weighing of powders, blending and compression, hence less cost), elimination of heat and moisture, prime particle dissociation, and physical stability.

In addition to the assignee of the subject application, Biovail Laboratories, current manufacturers of rapidly disintegrating or dissolving solid dose oral formulations include Cima Labs, Prographarm/Ethypharm, R. P. Scherer, and Yamanouchi-Shaklee. All of these manufacturers market different types of rapidly dissolving solid oral dosage forms.

Cima Labs markets OraSolv™, which is an effervescent direct compression tablet purportedly having an oral dissolution time of five to thirty seconds, and DuraSolv™, which is a direct compression tablet having a taste-masked active agent and a purported oral dissolution time of 15 to 45 seconds. Cima's U.S. Pat. No. 5,607,697, for “Taste Masking Microparticles for Oral Dosage Forms,” describes a solid dosage form consisting of coated microparticles that disintegrate in the mouth. The microparticle core has a pharmaceutical agent and one or more sweet-tasting compounds having a negative heat of solution selected from mannitol, sorbitol, a mixture of an artificial sweetener and menthol, a mixture of sugar and menthol, and methyl salicylate. The microparticle core is coated, at least partially, with a material that retards dissolution in the mouth and masks the taste of the pharmaceutical agent. The microparticles are then compressed to form a tablet. Other excipients can also be added to the tablet formulation.

WO 98/46215 for “Rapidly Dissolving Robust Dosage Form,” assigned to Cima Labs, is directed to a hard, compressed, fast melt formulation having an active ingredient and a matrix of at least a non-direct compression filler and lubricant. A non-direct compression filler is typically not free-flowing, in contrast to a direct compression (DC grade) filler, and usually requires additionally processing to form free-flowing granules.

Cima also has U.S. patents and international patent applications directed to effervescent dosage forms (U.S. Pat. Nos. 5,503,846, 5,223,264, and 5,178,878) and tableting aids for rapidly dissolving dosage forms (U.S. Pat. Nos. 5,401,513 and 5,219,574), and rapidly dissolving dosage forms for water soluble drugs (WO 98/14179 for “Taste-Masked Microcapsule Composition and Methods of Manufacture”).

Prographarm/Ethypharm markets Flashtab™, which is a fast melt tablet having a disintegrating agent such as carboxymethyl cellulose, a swelling agent such as a modified starch, and a taste-masked active agent. The tablets have a purported oral disintegration time of under one minute (U.S. Pat. No. 5,464,632).

R. P. Scherer markets Zydis™, which is a freeze-dried tablet having an oral dissolution time of 2 to 5 seconds. Lyophilized tablets are costly to manufacture and difficult to package because of the tablets sensitivity to moisture and temperature. U.S. Pat. No. 4,642,903 (R. P. Scherer Corp.) refers to a fast melt dosage formulation prepared by dispersing a gas throughout a solution or suspension to be freeze-dried. U.S. Pat. No. 5,188,825 (R. P. Scherer Corp.) refers to freeze-dried dosage forms prepared by bonding or complexing a water-soluble active agent to or with an ion exchange resin to form a substantially water insoluble complex, which is then mixed with an appropriate carrier and freeze dried. U.S. Pat. No. 5,631,023 (R. P. Scherer Corp.) refers to freeze-dried drug dosage forms made by adding xanthan gum to a suspension of gelatin and active agent. U.S. Pat. No. 5,827,541 (R. P. Scherer Corp.) discloses a process for preparing solid pharmaceutical dosage forms of hydrophobic substances. The process involves freeze-drying a dispersion containing a hydrophobic active ingredient and a surfactant, in a non-aqueous phase; and a carrier material, in an aqueous phase.

Yamanouchi-Shaklee markets Wowtab™, which is a tablet having a combination of a low moldability and a high moldability saccharide. U.S. patents covering this technology include U.S. Pat. No. 5,576,014 for “Intrabuccally Dissolving Compressed Moldings and Production Process Thereof,” and U.S. Pat. No. 5,446,464 for “Intrabuccally Disintegrating Preparation and Production Thereof.”

Other companies owning rapidly dissolving technology include Janssen Pharmaceutica. U.S. patents assigned to Janssen describe rapidly dissolving tablets having two polypeptide (or gelatin) components and a bulking agent, wherein the two components have a net charge of the same sign, and the first component is more soluble in aqueous solution than the second component. See U.S. Pat. No. 5,807,576 for “Rapidly Dissolving Tablet;” U.S. Pat. No. 5,635,210 for “Method of Making a Rapidly Dissolving Tablet;” U.S. Pat. No. 5,595,761 for “Particulate Support Matrix for Making a Rapidly Dissolving Tablet;” U.S. Pat. No. 5,587,180 for “Process for Making a Particulate Support Matrix for Making a Rapidly Dissolving Tablet;” and U.S. Pat. No. 5,776,491 for “Rapidly Dissolving Dosage Form.”

Eurand America, Inc. has U.S. patents directed to a rapidly dissolving effervescent composition having a mixture of sodium bicarbonate, citric acid, and ethylcellulose (U.S. Pat. Nos. 5,639,475 and 5,709,886).

L.A.B. Pharmaceutical Research owns U.S. patents directed to effervescent-based rapidly dissolving formulations having an effervescent couple of an effervescent acid and an effervescent base (U.S. Pat. Nos. 5,807,578 and 5,807,577).

Schering Corporation has technology relating to buccal tablets having an active agent, an excipient (which can be a surfactant) or at least one of sucrose, lactose, or sorbitol, and either magnesium stearate or sodium dodecyl sulfate (U.S. Pat. Nos. 5,112,616 and 5,073,374).

Laboratoire L. LaFon owns technology directed to conventional dosage forms made by lyophilization of an oil-in-water emulsion in which at least one of the two phases contains a surfactant (U.S. Pat. No. 4,616,047). For this type of formulation, the active ingredient is maintained in a frozen suspension state and is tableted without micronization or compression, as such processes could damage the active agent.

Takeda Chemicals Inc., Ltd. owns technology directed to a method of making a fast dissolving tablet in which an active agent and a moistened, soluble carbohydrate are compression molded into a tablet, followed by drying of the tablets.

Biovail Corporation (the parent of the assignee of the subject application) markets Flash Dose™, which is a direct compression tablet containing a processed excipient called Shearform™. Shearform™ is a floss type substance of mixed polysaccharides converted to amorphous fibers. U.S. patents describing this technology include U.S. Pat. No. 5,871,781 for “Apparatus for Making Rapidly Dissolving Dosage Units;” U.S. Pat. No. 5,869,098 for “Fast-Dissolving Comestible Units Formed Under High-Speed/High-Pressure Conditions;” U.S. Pat. Nos. 5,866,163, 5,851,553, and 5,622,719, all for “Process and Apparatus for Making Rapidly Dissolving Dosage Units and Product Therefrom;” U.S. Pat. No. 5,567,439 for “Delivery of Controlled-Release Systems;” and U.S. Pat. No. 5,587,172 for “Process for Forming Quickly Dispersing Comestible Unit and Product Therefrom.”

One way to provide self-binding flowable formulations is to formulate using Shearform™ matrices or flosses. These matrices result when using certain processing techniques, such as the following: U.S. Pat. No. 5,587,172, incorporated herein by reference, discusses the use of flash heat techniques to produce sucrose-containing shearform flosses, which are then processed to yield quick-dissolving tablets.

The use of shearform matrices for forming comestible units is described in WO95/34290 (published Dec. 21, 1995) from co-assigned PCT application No. PCT/US95/07144, filed Jun. 6, 1995. This case discloses a quick dissolving tablet which is formed by: (1) using flash-flow technology to provide a shearform matrix; (2) combining the partially recrystallized shearform matrix with an additive to form flowable, compactible particulate blends; and (3) compacting the blends at relatively low pressures to produce dosage forms, such as tablets.

Additionally, PCT publication WO 95/34293 (published Dec. 21, 1995) from co-assigned PCT Application No. PCT/US95/07194, filed Jun. 6, 1995, discloses a process and apparatus for making rapidly dissolving dosage forms by flash-flow processing. In this PCT application, a shearform matrix is formed by the flash-flow process, the shearform matrix is combined with an additive, and the matrix is molded to make a unit dosage form.

Co-owned U.S. patent application Ser. No. 08/915,068, filed Aug. 20, 1997, now U.S. Pat. No. 5,840,331; and Ser. No. 09/132,986, filed Aug. 12, 1998, now U.S. Pat. No. 6,048,541, describe tablet formulations derived from saccharide-based carriers in which the use of a unique combination of feedstock ingredients yields self-binding, flowable matrices and tablet compositions. This combination—which uses a blend of sugar alcohols, i.e., sorbitol and xylitol—is superior to glycerine in providing cohesive properties and flowability.

Shapeable, preferably tabletable, compositions derived from partially hygroscopic matrices containing these sugar alcohols are useful—in the presence of tableting aids and crystallization promoters—in both high—and low-pressure tableting processes. Tablets and other dosage forms, e.g., lozenges, made therefrom rapidly dissolve when placed in the mouth, generally in less than 30 seconds.

The production of microspheres containing active agent(s) is described in co-owned U.S. Pat. No. 5,683,720, incorporated herein by reference. The patent deals with the use of Liquiflash™ processing to spheronize compositions containing one or more active agents.

Co-owned U.S. Pat. No. 6,165,512 provides compositions and shaped oral dosage forms made therefrom having improved properties. Among those properties are improved processability before shaping and enhanced dissolution and taste-masking properties when the dosage forms are used. The compositions of the '512 patent are based on matrices, or flosses, which comprise at least one sugar alcohol, which matrices are generally considered “single floss” or “unifloss” systems. These systems are exemplified by xylitol-containing shearform matrixes, or flosses, containing a carrier and two or more sugar alcohols.

Various ingredients, such as coated microspheres containing active agent(s), are added, in suitable amounts, to the compositions of the present invention after the matrices are collected and chopped, but before they are shaped, e.g., by tabletting.

Highly useful dosage forms result when microspheres made from compositions containing active agents, solubilizers and spheronization aids are coated with taste-masking agents, then combined with flosses and conventional pharmaceutical ingredients. The resultant tablets enjoy the processing ease associated with the use of glycerine-free flosses and the taste and release properties associated with coated microspheres.

The above mentioned existing quick dissolve technologies present numerous limitations. The above mentioned Prographarm (Ethypharm) dosage forms require relatively high levels of super disintegrant which complicates their use and limits their friability and hardness thereby requiring specialized packaging. Similarly, the Cima dosage forms require effervescent excipients which also reduces their friability and hardness qualities. The RP Scherer, Yamanouchi and Takada technologies employ complicated processing techniques (i.e. lyophilization, solvents with heat treatment or drying). Those techniques increase the cost associated with the formation of the dosage forms on a large scale.

While Shearform™ matrices are an advance in the art, they also involve an increased cost associated with the processing of the floss matrix which limits their use at a large scale. As well, these amorphous matrices require specialized robotic tableting equipment and generally do not provide friability and hardness properties required for bulk packaging such as in bottles.

As indicated above, disintegrants are often included to ensure that the tablet has an acceptable rate of disintegration. Typical disintegrants include starch derivatives and salts of carboxymethylcellulose. Thus, there still exists a need for non-sticking tabletable compositions which, can be used to make fast-dissolving, pleasant tasting dosage forms at a low cost and without the need for excessive amounts of super disintegrant or complicated processing equipment.

### SUMMARY OF THE INVENTION

The present invention is based on the unexpected discovery that quick dissolve Flashdose™ tablets can be provided without the need for floss matrices. The inventors have unexpectedly discovered that under certain processing conditions, direct compression of Liquiflash™ microspheres, in particular microspheres prepared according to co-owned U.S. patent application Ser. No. 09/179,926 provides quick dissolve dosage without the need for a floss matrix or super disintegrant as defined below or with quantities of super disintegrant that are well below the levels employed with the dosage forms discussed in the background section.

In addition to the fast dissolve properties provided by the compositions of the invention, other advantages of the invention include the use of appropriate excipient mass (e.g., directly compressible inorganic salt; cellulose derivatives, etc.), which in turn facilitates the processing of the composition and eliminates the need for complex processing equipment. The components of the composition of the invention and the processing methods associated therewith allow for substantially lowering the cost associated with the production of the quick dissolve dosage forms of the invention which in turn facilitates their use at a large scale. Also, the simplicity of the excipients and the techniques employed in forming the dosage forms of the invention reduces the number of steps in manufacturing the dosage forms, thereby drastically reducing the opportunities for contamination and other quality impacting deleterious effects. The dosage forms of the invention are also advantageous in that higher loads of active agent can be obtained.

As well, the compositions and dosage forms of the invention are greatly advantageous in that packaging is simplified. In fact, the present invention provides a unique combination of materials and processing techniques that allows the packaging of quick dissolve dosage forms in recipients as commonly used and easy to access as prescription or over the counter bottles and blister packaging. The simpler packaging advantages of the composition of the invention are due at least in part to the improved friability and hardness obtained with the quick dissolve dosage forms of the invention.

In one embodiment, the invention provides a composition useful for making oral dosage forms capable of dissolving in the mouth in less than 40 seconds without the need for a conventional super disintegrant and having a friability of less than 1%; wherein the composition comprises drug-containing liquiflash particles and an excipient mass. Preferred excipient mass comprises a directly compressible inorganic salt, a cellulose derivative or a mixture of a directly compressible salt and a cellulose derivative. Preferably, the liquiflash particles and the mass of excipient are combined in proportions such that the active ingredient remains substantially within the microspheres when the composition is compressed to obtain a dosage form having a hardness of about 20 N to 50 N. The improved hardness and friability are obtained due to the discovery that the combination of the microspheres and the excipient mass allows for higher compression force.

The liquiflash particles are preferably coated with at least one taste-masking coating. The coating preferably contains at least one cellulosic polymer. To improve the dissolution properties of the dosage form of the invention the composition may further comprises microcrystalline cellulose which facilitates disintegration in the mouth without having super disintegrant properties. A preferred linear polyol comprises mannitol, alone or in combination with sorbitol.

A preferred embodiment of the invention provides a composition useful for making oral dosage forms capable of dissolving in the mouth in less than 30 seconds and having a friability of less than 1%; wherein the composition comprises liquiflash particles containing at least one bioaffecting agent and a combination of at least one solubilizer and at least one spheronization aid, said liquiflash particles being coated after spheronization; a mass comprising an excipient mass and less than 2.5% by weight of a super disintegrant.

As indicated below, the compositions of the invention can be successfully employed to prepare oral dosage forms of a variety of active agents. Particularly preferred active agents include fluoxetine; paroxetine and zolpidem.

### DETAILED DESCRIPTION OF THE INVENTION

The invention is concerned with bio-affecting microparticles produced from compositions containing a unique combination of ingredients. The composition, the microparticles, their production and comestible units containing them are disclosed.

Unless stated otherwise, all percentages recited herein are weight percentages, based on total composition weight.

### I. Disintegrants and Super Disintegrants:

A disintegrant is an excipient which is added to a tablet or capsule blend to aid in the break up of the compacted mass when it is put into a fluid environment. This is especially important for immediate release products where rapid release of drug substance is required. A disintegrant can be added to a powder blend for direct compression or encapsulation. It can also be used with products that are wet granulated. In wet granulation formulations, the disintegrant is normally effective when incorporated into the granule (intragranularly). However, it may be more effective if added 50% intragranularly, and 50% extra-granularly (i.e., in the final dry mixture). While there are some tablet fillers (e.g., starch and microcrystalline cellulose) which aid in disintegration, there are more effective agents referred to as superdisintegrants. Some superdisintegrants and their properties are listed below.

| | |
|---|---|
| Crosscarmelose sodium | High swelling capacity, effective at low concentrations (0.5-2.0% |
| | but can be used up to 5.0%). |
| Crospovidone | Completely insoluble in water. Rapidly disperses and swells in |
| | water, but does not gel even after prolonged exposure. Greatest rate |
| | of swelling compared to other disintegrants. Greater surface area to |
| | volume ratio than other disintegrants. Recommended concentration: |
| | 1 to 3% |
| | Available in micronized grades if needed to improve uniform |
| | dispersion in the powder blend. |
| Sodium Starch Glycolate | Absorbs water rapidly, resulting in swelling which leads to rapid |
| | disintegration of tablets and granules. Recommended concentration: |
| | 1.0-4.0% but may need to use up to 6.0%. Gels on prolonged |
| | exposure to water. High concentrations may cause gelling and loss |
| | of disintegration. |

A super disintegrant according to the invention is a disintegrant that has a Eq. Moisture content at 25C/90% RH of over 50%. A list of exemplary disintegrants, super disintegrants and other formulations with some disintegrant qualities are provided below:

Superdisintegrants and Disintegrants

| | | | | | Eq. | |
|---|---|---|---|---|---|---|
| | | | | | Moisture | |
| Brand | Common | | Functional | | content at | |
| name | name | Classification | Category | Properties | 25 C./90% RH | Typical uses |
| CL- | Crospovidone | Polyvinylpolypyrrolidone | Tablet | Hygroscopic | 62% | Disintegrant in |
| Kollidon | | | super | Swelling- | | dry |
| | | | disintegrant | 18% in 10 s, | | and wet |
| | | | | 45% in 20 s | | granulation |
| Ac- | Croscarmellose | Cellulose, | Tablet | Hygroscopic | 88% | Disintegrant for |
| Disol | sodium | carboxymethyl | and | Wicking | | capsules, tablets |
| Primellose | | ether, | capsule | and | | and granules |
| | | sodium salt, | super | swelling- | | |
| | | crosslinked | disintegrant | 12% in 10 s, | | |
| | | | | 23% in 20 s | | |
| Explotab | Sodium | Sodium | Tablet | Swelling | | Disintegrant in |
| Primojel | starch | carboxymethyl | and | capacity: in | | dry and wet |
| | glycolate | starch | capsule | water swells | | granulation |
| | | | super | up to 300 | | |
| | | | disintegrant | times its | | |
| | | | | volume | | |
| Explotab | Sodium | (Cross | Super | Swells to | | Disintegration |
| V17 | starch | linked low | disintegrant | greater | | and dissolution |
| | glycolate | substituted | | extent than | | aid. Not for use |
| | | carboxymethyl | | explotab | | in wet |
| | | ether)Sodium | | | | granulation |
| | | carboxymethyl | | | | |
| | | starch | | | | |
| Explotab | Sodium | (Cross | Super | | | Designed for |
| CLV | starch | linked low | disintegrant | | | wet granulation |
| | glycolate | substituted | | | | that utilize high |
| | | carboxymethyl | | | | shear equipment |
| | | ether)Sodium | | | | |
| | | carboxymethyl | | | | |
| | | starch, | | | | |
| | | highly cross | | | | |
| | | linked | | | | |
| L-HPC | Hydroxypropyl | Cellulose, | Tablet | Hygroscopic | 37% | Tablet |
| | cellulose, | 2- | and | Swelling- | | disintegrant, |
| | low- | hydroxypropyl | capsule | 13% in 10 s, | | binder in wet |
| | substituted | ether | disintegrant, | 50% in 20 s | | granulation |
| | | (low | tablet | | | |
| | | substituted) | binder | | | |
| Amberlite | Polacrilin | Cation | Tablet | Swelling | | Tablet |
| IRP | Potassium | exchange | disintegrant | ability | | disintegrant |
| 88 | | resin | | | | |
| Starch | Starch, | Pregelatinized | Tablet | Hygroscopic | 22% | Capsule and |
| 1500 | pregelatinized | starch | and | | | tablet binder, |
| | | | capsule | | | diluent, |
| | | | diluent, | | | disintegrant |
| | | | disintegrant, | | | |
| | | | tablet | | | |
| | | | binder | | | |
| Avicel | Microcrystalline | Cellulose | Tablet | Hygroscopic | 18% | Binder/diluent, |
| | cellulose | | and | Swelling- | | has also some |
| | | | capsule | 12% in 10 s, | | lubricant and |
| | | | diluent, | 18% in 20 s | | disintegrant |
| | | | tablet | | | properties |
| | | | disintegrant | | | |

### II. Compositions

The compositions of the invention employ optional excipients with (a) a bioaffecting agent and (b) one or more processing aids.

A. Bio-Affecting Agents

The active ingredients useful herein can be selected from a large group of therapeutic agents. Respective classes include those in the following therapeutic categories: ace-inhibitors; alkaloids; antacids; analgesics; anabolic agents; anti-anginal drugs; anti-allergy agents; anti-arrhythmia agents; antiasthmatics; antibiotics; anticholesterolemics; anticonvulsants; anticoagulants; antidepressants; antidiarrheal preparations; anti-emetics; antihistamines; antihypertensives; anti-infectives; anti-inflammatories; antilipid agents; antimanics; anti-migraine agents; antinauseants; antipsychotics; antistroke agents; antithyroid preparations; anabolic drugs; antiobesity agents; antiparasitics; antipsychotics; antipyretics; antispasmodics; antithrombotics; antitumor agents; antitussives; antiulcer agents; anti-uricemic agents; anxiolytic agents; appetite stimulants; appetite suppressants; beta-blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypoglycemic agents; ion-exchange resins; laxatives; migraine treatments; mineral supplements; mucolytics, narcotics; neuroleptics; neuromuscular drugs; non-steroidal anti-inflammatories (NSAIDs); nutritional additives; peripheral vasodilators; polypeptides; prostaglandins; psychotropics; renin inhibitors; respiratory stimulants; sedatives; steroids; stimulants; sympatholytics; thyroid preparations; tranquilizers; uterine relaxants; vaginal preparations; vasoconstrictors; vasodilators; vertigo agents; vitamins; wound healing agents; and others. Active agents which may be used in the invention include: acetaminophen; acetic acid; acetylsalicylic acid, including its buffered forms; acrivastine; albuterol and its sulfate; alcohol; alkaline phosphatase; allantoin; aloe; aluminum acetate, carbonate, chlorohydrate and hydroxide; alprazolam; amino acids; aminobenzoic acid; amoxicillin; ampicillin; amsacrine; amsalog; anethole; ascorbic acid; aspartame; astemizole; atenolol; azatidine and its maleate; bacitracin; balsam peru; BCNU (carmustine); beclomethasone dipropionate; benzocaine; benzoic acid; benzophenones; benzoyl peroxide; benzquinamide and its hydrochloride; bethanechol; biotin; bisacodyl; bismuth subsalicylate; bornyl acetate; bromopheniramine and its maleate; buspirone; caffeine; calamine; calcium carbonate, casinate and hydroxide; camphor; captopril; cascara sagrada; castor oil; cefaclor; cefadroxil; cephalexin; centrizine and its hydrochloride; cetyl alcohol; cetylpyridinium chloride; chelated minerals; chloramphenicol; chlorocyclizine hydrochloride; chlorhexidine gluconate; chloroxylenol; chloropentostatin; chlorpheniramine and its maleates and tannates; chlorpromazine; cholestyramine resin; choline bitartrate; chondrogenic stimulating protein; cimetidine and its hydrochloride; cinnamedrine hydrochloride; citalopram; citric acid; clarithromycin; clemastine and its fumarate; clonidine and its hydrochloride salt; clofibrate; cocoa butter; cod liver oil; codeine and its fumarate and phosphate; cortisone acetate; ciprofloxacin HCl; cyanocobalamin; cyclizine hydrochloride; cyproheptadine and its hydrochloride; danthron; dexbromopheniramine maleate; dextromethorphan and its hydrohalides; diazepam; dibucaine; dichloralphenazone; diclofen and its alkali metal sales; diclofenac sodium; digoxin; dihydroergotamine and its hydrogenates/mesylates; diltiazem; dimethicone; dioxybenzone; diphenhydramine and its citrate; diphenhydramine and its hydrochloride; divalproex and its alkali metal salts; docusate calcium, potassium, and sodium; doxycycline hydrate; doxylamine succinate; dronabinol; efaroxan; enalapril; enoxacin; ergotamine and its tartrate; erythromycin; estropipate; ethinyl estradiol; ephedrine; epinephrine bitartrate; erythropoietin; eucalyptol; famotidine; fenoprofen and its metal salts; ferrous fumarate, gluconate and sulfate; fluoxetine; folic acid; fosphenytoin; 5-fluorouracil (5-FU); fluoxetine and its hydrochloride; flurbiprofen; furosemide; gabapentan; gentamicin; gemfibrozil; glipizide; glycerine; glyceryl stearate; granisetron and its hydrochloride; griseofulvin; growth hormone; guafenesin; hexylresorcinol; hydrochlorothiazide; hydrocodone and its tartrates; hydrocortisone and its acetate; 8-hydroxyquinoline sulfate; hydroxyzine and its pamoate and hydrochloride salts; ibuprofen; indomethacin; inositol; insulin; iodine; ipecac; iron; isosorbide and its mono and dinitrates; isoxicam; ketamine; kaolin; ketoprofen; lactic acid; lanolin; lecithin; leuprolide acetate; lidocaine and its hydrochloride salt; lifinopril; liotrix; loratadine; lovastatin; luteinizing hormore; LHRH (lutenizing hormone replacement hormone); magnesium carbonate, hydroxide, salicylate, and trisilicate; meclizine and its hydrochloride; mefenamic acid; meclofenamic acid; meclofenamate sodium; medroxyprogesterone acetate; methenamine mandelate; menthol; meperidine hydrochloride; metaproterenol sulfate; methscopolamine and its nitrates; methsergide and its maleate; methyl nicotinate; methyl salicylate; methyl cellulose; methsuximide; metoclopramide and its halides/hydrates; metronidazole and its hydrochloride; metoprotol tartrate; miconazole nitrate; mineral oil; minoxidil; morphine; naproxen and its alkali metal sodium salts; nifedipine; neomycin sulfate; niacin; niacinamide; nicotine; nicotinamide; nimesulide; nitroglycerine; nonoxynol-9; norethindrone and its acetate; nystatin; octoxynol; octoxynol-9; octyl dimethyl PABA; octyl methoxycinnamate; omega-3 polyunsaturated fatty acids; omeprazole; ondansetron and its hydrochloride; oxolinic acid; oxybenzone; oxtriphylline; para-aminobenzoic acid (PABA); padimate-O; paramethadione; pentastatin; peppermint oil; pentaerythritol tetranitrate; pentobarbital sodium; perphenazine; phenelzine sulfate; phenindamine and its tartrate; pheniramine maleate; phenobarbital; phenol; phenolphthalein; phenylephrine and its tannates and hydrochlorides; phenylpropanolamine and its hydrochloride salt; phenytoin; pirmenol; piroxicam and its salts; polymicin B sulfate; potassium chloride and nitrate; prazepam; procainamide hydrochloride; procaterol; promethazine and its hydrochloride; propoxyphene and its hydrochloride and napsylate; pramiracetin; pramoxine and its hydrochloride salt; prochlorperazine and its maleate; propanolol and its hydrochloride; promethazine and its hydrochloride; propanolol; pseudoephedrine and its sulfates and hydrochorides; pyridoxine; pyrolamine and its hydrochlorides and tannates; quinapril; quinidine gluconate and sulfate; quinestrol; ralitoline; ranitadine; resorcinol; riboflavin; salicylic acid; scopolamine; sesame oil; shark liver oil; simethicone; sodium bicarbonate, citrate, and fluoride; sodium monofluorophosphate; sucralfate; sulfanethoxazole; sulfasalazine; sulfur; sumatriptan and its succinate; tacrine and its hydrochloride; theophylline; terfenadine; thiethylperazine and its maleate; timolol and its maleate; thioperidone; tramadol; trimetrexate; triazolam; tretinoin; tetracycline hydrochloride; tolmetin; tolnaftate; triclosan; trimethobenzamide and its hydrochloride; tripelennamine and its hydrochloride; tripolidine hydrochloride; undecylenic acid; vancomycin; verapamil HCI; vidaribine phosphate; vitamins A, B, C, D, BI, B2, B6, B,2, E, and K; witch hazel; xylometazoline hydrochloride; zinc; zinc sulfate; zinc undecylenate. Mixtures and pharmaceutically acceptable salts of these and other actives can be used.

Particularly useful active agents are sparingly soluble solid agents whose dissolution and release properties are enhanced by the solubilizing agents used herein. These agents include HZ antagonists, analgesics, including non-steroidal anti-inflammatory drugs (NSAIDs), anticholesterolemics, anti-allergy agents, and anti-migraine agents.

Analgesics include aspirin, acetaminophen, acetaminophen plus caffeine, and non-steroidal anti-inflammatory drugs (NSAIDS), e.g., ibuprofen and nimesulide.

Useful NSAIDs include ibuprofen; diclofenac and its alkali metal salts; fenoprofen and its metal salts; fluriprofen; ketoprofen; naproxen and its alkali metal salts; nimesulide; and piroxicam and its salts.

H₂-antagonists which are contemplated for use in the present invention include cimetidine, ranitidine hydrochloride, famotidine, nizatidine, ebrotidine, mifentidine, roxatidine, pisatidine and aceroxatidine.

Useful anti-allergy agents include hydricodone and its tartrates; clemastine and its fumarate; azatadine and its maleate; acetaminophen; hydroxyzine and its pamoate and hydrochloride salts; chlorpheniramine and its maleates and tannates; pseudoephedrine and its sulfates and hydrochlorides; bromopheniramine and its maleate; dextromethorphan and its hydrohalides; loratadine; phenylephrine and its tannates and hydrochlorides; methscopolamine and its nitrates; phenylpropanolamine and its hydrochlorides; codeine and its hydrochloride; codeine and its phosphate; terfenadine; acrivastine; astemizole; cetrizine and its hydrochloride; phenindamine and its tartrate; tripelennamine and its hydrochloride; cyproheptadine and its hydrochloride; promethazine and its hydrochloride; and pyrilamine and its hydrochlorides and tannates.

Useful antimigraine agents include divalproex and its alkali metal salts; timolol and its maleate; propanolol and its hydrohalides; ergotamine and its tartrate; caffeine; sumatriptan and its succinate; dihydroergotamine, its hydrogenates/mesylates; methsergide and its maleate; isometheptene mucate; and dichloralphenazone.

Another class of drugs which can be used are antiemetics. Useful antiemetics include: meclizine and its hydrochloride; hydroxyzine and its hydrochloride and pamoate; diphenhydramine and its hydrochloride; prochlorperazine and its maleate; benzquinamide and its hydrochloride; granisetron and its hydrochloride; dronabinol; bismuth subsalicylate; promethazine and its hydrochloride; metoclopramide and its halides/hydrates; chlorpromazine; trimethobenzamide and its hydrochloride; thiethylperazine and its maleate; scopolamine; perphenazine; and ondansetron and its hydrochloride.

Other active ingredients for use in the present invention include antidiarrheals such as immodium AD, antihistamines, antitussives, decongestants, vitamins, and breath fresheners. Also contemplated for use herein are anxiolytics such as Xanax; antipsychotics such as Clozaril and Haldon; antihistamines such as Seldane, Hismanal, Relafen, and Tavist; antiemetics such as Kytril and Cesamet; bronchodilators such as Bentolin, Proventil; antidepressants such as Prozac, Zoloft, and Paxil; antimigranes such as Imigran, ACE-inhibitors such as Vasotec, Capoten and Zestril; Anti-Alzheimers agents such as Nicergoline; and Call-Antagonists such as Procardia, Adalat, and Calan.

Among the anticholesterolemics, the statins, e.g., lovastatin, provastatin and the like are notable.

Fluoxetine, paroxetine and zolpidem are preferred active agents.

Combinations of various types of drugs, as well as combinations of individual drugs, are contemplated.

B. Processing Aids

The processing aids of the invention include high molecular weight polyethylene glycols (PEG's) and/or polyethylene glycol glyceryl esters. When microspheres are made, these materials can be called “spheronization aids.”

By “high molecular weight polyethylene glycols (PEG),” applicants mean PEG's having molecular weights of about 3,000 to about 8,000. “PEG 4600,” having an average molecular weight of about 4400 to 4800, is a preferred material. Mixtures can be used.

In chemical terms, useful PEGS are those molecules having the structural formula HOCH₂(CH₂OCH²)^{m} CH₂OH, wherein m is the average number of oxyethylene groups. PEG's used for this invention are those in which m is from about 0 to about 13.

Useful PEGS are solids. They are discussed on pages 355-361 of the Handbook of Pharmaceutical Excipients, 2^{nd} ed. (1994).

The polyethylene glycol glyceryl esters useful herein are selected from those containing about 30 to about 35 oxyethylene groups. Polyethylene glycol 32 glyceryl ester sold as “GELUCIRE 50/13” by Gattefosse S.A. of France is a preferred ester. Mixtures are operable.

The amounts of ingredients used in the compositions are generally within those shown in the following table.

| | Broad range | Narrow range | Preferred range |
|---|---|---|---|
| Bio-affecting agent(s) | 1-50% | 5-40% | 20-30% |
| PEG | 0-90% | 60-90% | 60-80% |
| Glyceryl ester | 0-60% | 1-10% | 2.5-7.5% |
| Excipient(s) | 0-98% | 10-50% | 10-30% |

### III. Processes

Useful processes for making the microparticles of the invention include liquiflash conditions as well as other thermoforming processes known in the art, e.g., extrusion. “Liquiflash conditions” are generally those under which the material, called a feedstock, is rapidly heated just to the point at which it undergoes intraparticulate flow and partially deforms or liquifies so that it can pass through openings in a suitable spinning device. The passage of the liquiflash particles through openings is in response to centrifugal forces within the spinning head, which forces “expel” the particles, as discrete solids out of the device and into the atmosphere. The expelled materials instantly reform into particles, without the application of external shaping forces, which particles have different morphologies from those of the feedstocks.

Applicants have found that one particular spinning device is highly useful in making the microspheres of the, invention. In U.S. Pat. No. 5,458,823, a spinning device is described which uses a spinning head including a base and a cover. A plurality of closely spaced heating elements are positioned between the base and cover, forming a barrier through which the material to be processed passes. In use, the head rotates and the heating elements are heated to temperatures that bring about liquiflash conditions in the materials being processed. As the spinning head rotates, the centrifugal force created by its rotation expels the material through spaces between the heating elements. The material forms discrete, generally spherical particles as it exits.

The production of microspheres for use in the subject invention may be optimized by the use of a V-groove insert inside the spinner head. The insert is described in pending U.S. patent application Ser. No. 08/874,515, filed Jun. 13, 1997 The insert has grooves therein, which grooves have a uniform depth and width through their length, so that highly uniform discrete microspheres or other particles are produced. Using this or a similar insert, the spinning device is operated at 50 to 75 Hz, at about 10 to 25% power, and at temperatures which yield liquiflash conditions.

It should be noted that “liquiflash conditions” vary with the properties of the material, or feedstock, being processed. Since the feedstocks contain many substances in varying amounts, the parameters need to yield “liquiflash conditions” for a particular mixture must be ascertained by processing small quantities or samples before processing large ones. Typically, the feedstocks contain active agent(s) and processing aids.

Among the co-assigned patents and patent applications which describe the preparations of microspheres containing bio-affecting agents re: U.S. Pat. No. 5,458,823; U.S. Pat. No. 5,0q,720; and U.S. Ser. No. 08/874,215, filed Jun. 13, 1997.

### III. Microparticles

While particulates made using various thermoprocessing technologies are useful, microspheres described below are preferred.

The microspheres or other particulates are generally solid spherical bodies of about 150 to about 250 microns mean particle diameter.

It is preferred that they be produced via a direct spheronization process, such as liquiflash or other suitable techniques. However, they may be made by physically altering the size and/or shape of non-spherical particles by extrusion/spheronization or melt granulation processes.

When microspheres are made by direct spheronization of compositions containing active agent(s), the fatty esters and optional emulsifiers/surfactants, the fatty esters function as spheronization aids.

The microspheres may be used as is, i.e., in powder or sachet products for delivering active agents. Alternatively, they may be used in the production of solid, liquid (suspensions), or semi-solid (e.g., gel-like) comestible units, etc. Tablets and capsules are preferred.

It is preferred that the microspheres of the invention be used in combination with. excipients which have been formed into floss or matrix particles. Useful flosses are generally made from saccharide based carriers. See U.S. Pat. Nos. 5,622,719 and 5,587,172.

Once the floss and microsphere ingredients are combined, they can be shaped into comestible units.

### IV. Coatings

One or both of the microspheres and the dosage units can be coated or encapsulated with at least one coating. Useful coating formulations contain polymeric ingredients as well as excipients conventionally employed in such coatings. The coatings are generally used for such purposes as taste-masking, controlling release and the like.

Useful taste-masking coatings can include (meth)acrylate/cellulosic polymers. Ethylcellulose (EC), hydroxypropylcellulose (HPC), hydroxypropylmethylcellulose (HPMC), and polymethacrylate polymers, such as Eudragit RS, Eudragit RL or mixtures thereof are useful. Preferred combinations include EC/HPC and Eudragit RS/Eudragit RL.

Controlled release coatings generally contain at least one of ethylcellulose (EC), hydroxypropylcellulose (HPC), hydroxypropylmethyl cellulose (HPMC), hydroxypropylmethylcellulose phthalate, cellulose acetate phthalate, and the like. The “Eudragits” designated as NE 300, RS, L 30 D, are useful. Mixtures are operable.

Coating levels of about 0 to about 150% are effective, with levels of about 5% to about 30% being preferred.

Coating devices include those conventionally used in pharmaceutical processing, with fluidized bed coating devices being preferred.

Formulations according to the invention are illustrated by the examples provided below, which should in no way limit the scope of the appended claims. The friability results shown below correspond to Drop tests conducted with a Roche drum equipped with two separated drums, the motor rotate the drum at 100 revolution/min. the actual drums is made from plexiglass and is separated into parts, the drum body and removable cover, which opens to fill, discharge and clean the drum. For the Abrasion tests one of the two drums is replaced with an abrasion drum.

### EXAMPLES

The examples and counterexamples provided below illustrate formulations and processing conditions for forming dosage forms according to the invention.

### Formulation No 1

CEFORM™ or other coated particle: 5-45% W/W, preferred 5-35%, (35-45% is fast tablet but gritty)

### Mannitol*: 29.1-77.1%

### Microcrystalline Cellulose**: 12-18%

### 1-HPC, LH-11: 2-4%

### Citric Acid: 1.5%

### Acesulfame K: 0.2%

### Magnasweet 100: 0.2%

### Flavor: 0.5%

### Syloid: 0.5%

### Pruv: 1.0%

### Formulation NO 2

CEFORM™ or other coated particle: 5-45% W/W, preferred 5-35%, (35-45% is fast tablet but gritty)

### Mannitol*: 29.1-77.1%

Microcrystalline Cellulose**: 12-18%, preferably 15%-18%

### Kollidon CL: 2-4%

### Citric Acid: 1.5%

### Acesulfame K: 0.2%

### Magnasweet 100: 0.2%

### Flavor: 0.5%

### Syloid: 0.5%

### Pruv: 1.0%

### Formulation No 3 (More Referred Platform):

CEFORM™ or other coated particle: 5-45% W/W, preferred 5-35%, (35-45% is fast tablet but gritty)

### Mannitol*: 27.1-83.6%

Microcrystalline Cellulose**: 5-20%, preferably 15-18%

### Kollidon CL: 2%

### 1-HPC, LH-11: 2%

### Citric Acid: 1.5%

### Acesulfame K: 0.2%

### Magnasweet 100: 0.2%

### Flavor: 0.5%

### Syloid: 0.5%

### Pruv: 1.0%

*Mannitols evaluated and found acceptable: Pearlitol 400DC, 300DC, Parteck M200, Parteck M300, Roquette Lab 3038. No differences were observed in disintegration time.
**Microcrystalline cellulose evaluated and found acceptable: Avicel PH 101, 102, 113, Prosolv 50, Prosolv 90. No differences were observed in disintegration time.

Other preferred formulations based on model drug fluoxetine:

| | Hardness | Disintegration | | |
|---|---|---|---|---|
| Formulation Lot# | (N) | time | Friability % | Comments |
| Fluoxetine TMMS: | 29.7 | Mouth: 10 s | 0.8 | Can be used with |
| 28.69 | | USP basket | | any drug |
| Pearlitol 400DC: | | rack assembly: | | |
| 48.41 | | 20 s | | |
| Avicel PH 101: 16.0 | | | | |
| L-HPC 11: 4.0 | | | | |
| Citric acid: 1.0 | | | | |
| AsesulK: 0.2 | | | | |
| Tangerine: 0.2 | | | | |
| Syloid: 0.5 | | | | |
| Pruv: 1.0 | | | | |
| Avicel PH101/L- | | | | |
| HPC11 ratio (80/20) | | | | |
| Lot#/mfg date: 1242- | | | | |
| 124 | | | | |
| 250 g batch/11 mm | | | | |
| Flat Face Radial | | | | |
| Edge/450 mg | | | | |
| Fluoxetine TMMS: | 34.0 | Mouth: 10 s | 0.8 | Can be used with |
| 28.69 | | USP basket | | any drug |
| Pearlitol 400DC: | | rack assembly: | | |
| 48.41 | | 20 s | | |
| Avicel PH 101: 18.0 | | | | |
| L-HPC 11: 2.0 | | | | |
| Citric acid: 1.0 | | | | |
| AsesulK: 0.2 | | | | |
| Tangerine: 0.2 | | | | |
| Syloid: 0.5 | | | | |
| Pruv: 1.0 | | | | |
| Avicel PH101/L- | | | | |
| HPC11 (90/10) ratio | | | | |
| Lot#/mfg date: 1242- | | | | |
| 125 | | | | |
| 250 g batch/11 mm | | | | |
| Flat Face Radial | | | | |
| Edge/450 mg | | | | |
| Fluoxetine TMMS: | 29.5 | Mouth: 10 s, | 0.3 | Can be used with |
| 28.69 | 24.4 | 15 s, 20 s, 10 s, | 0.3 | any drug |
| Pearlitol 400DC: | 28.4 | 10 s | 0.2 | |
| 51.41 | 26.0 | USP basket | 0.2 | |
| Avicel PH 101: 15.0 | 28.3 | rack assembly: | 0.4 | |
| L-HPC 11: 2.0 | | 15 s, 20 s, —, | | |
| Citric acid: 1.0 | | 19 s, — | | |
| AsesulK: 0.2 | | | | |
| Tangerine: 0.2 | | | | |
| Syloid: 0.5 | | | | |
| Pruv: 1.0 | | | | |
| *can be | | | | |
| Avicel 113, 1242- | | | | |
| 140 | | | | |
| Avicel 102, 1242- | | | | |
| 139 | | | | |
| Prosolv 50, 1242- | | | | |
| 138 | | | | |
| Prosolv 90, 1242- | | | | |
| 137 | | | | |
| Lot#/mfg date: 1242- | | | | |
| 135, 140, 139, 138, | | | | |
| 137 | | | | |
| 250 g batch/11 mm | | | | |
| Flat Face Radial | | | | |
| Edge/450 mg | | | | |
| Fluoxetine TMMS: | 28.4 | Mouth: 15 s. | 0.5 | Can be used with |
| 28.69 | | Good tablets | | any drug except the |
| Advantose 100: | | No significant | | drugs that have |
| 12.85 | | difference | | amine group. |
| Pearlitol 400DC: | | between 1242- | | |
| 38.56 | | 147 | | |
| Avicel PH 101: 15.0 | | USP basket | | |
| L-HPC 11: 2.0 | | rack assembly: | | |
| Citric acid: 1.0 | | 19 s | | |
| AsesulK: 0.2 | | | | |
| Tangerine: 0.2 | | | | |
| Syloid: 0.5 | | | | |
| Pruv: 1.0 | | | | |
| Advantose | | | | |
| 100/Pearlitol 400DC | | | | |
| (25/75) ratio | | | | |
| Lot#/mfg date: 1242- | | | | |
| 148/ | | | | |
| Feb. 4, 2002 | | | | |
| 250 g batch/11 mm | | | | |
| Flat Face Radial | | | | |
| Edge/450 mg | | | | |
| Fluoxetine TMMS: | 33.9 | Mouth: 7-10 s | 0.6 | Can be used with |
| 28.69 | | very fast tablet | | any drug |
| Pearlitol 400DC: | | USP basket | | |
| 51.41 | | rack assembly: | | |
| Avicel PH 101: 15.0 | | 31 s | | |
| Kollidon CL: 2.0 | | | | |
| Citric acid: 1.0 | | | | |
| AsesulK: 0.2 | | | | |
| Syloid: 0.5 | | | | |
| Tangerine: 0.2 | | | | |
| Pruv: 1.0 | | | | |
| Lot#/mfg date: 1242- | | | | |
| 152/ | | | | |
| Feb. 5, 2002 | | | | |
| 250 g batch/11 mm Flat | | | | |
| Face Radial | | | | |
| Edge/450 mg | | | | |
| Fluoxetine TMMS: | 30.8 | Mouth: 10 s | 0.2 | Can be used with |
| 28.69 | | very fast tablet | | any drug except the |
| Pearlitol 400DC: | | USP basket | | drugs that have |
| 38.56 | | rack assembly: | | amine group. |
| Advantose 100: 12.85 | | 19 s | | |
| Avicel PH 101: 15.0 | | | | |
| Kollidon CL: 2.0 | | | | |
| Citric acid: 1.0 | | | | |
| AsesulK: 0.2 | | | | |
| Syloid: 0.5 | | | | |
| Tangerine: 0.2 | | | | |
| Pruv: 1.0 | | | | |
| Lot#/mfg date: 1242- | | | | |
| 153/ | | | | |
| Feb. 5, 2002 | | | | |
| 250 g batch/11 mm Flat | | | | |
| Face Radial | | | | |
| Edge/450 mg | | | | |
| Fluoxetine TMMS: | 29.4 | Mouth: 10 s | 0.6 | Can be used with |
| 28.69 | | very fast tablet, | | any drug |
| Pearlitol 400DC: | | no difference | | |
| 49.41 | | between 1242- | | |
| Avicel PH 101: 15.0 | | 154 & 140 | | |
| Kollidon CL: 2.0 | | batches | | |
| L-HPC 11: 4.0 | | USP basket | | |
| Citric acid: 1.0 | | rack assembly: | | |
| AsesulK: 0.2 | | 23 s | | |
| Syloid: 0.5 | | | | |
| Tangerine: 0.2 | | | | |
| Pruv: 1.0 | | | | |
| Lot#/mfg date: 1242- | | | | |
| 157/ | | | | |
| Feb. 6, 2002 | | | | |
| 250 g batch/11 mm Flat | | | | |
| Face Radial | | | | |
| Edge/450 mg | | | | |
| Fluoxetine TMMS: | 33.1 | Mouth: 12-15 s | 0.6 | Can be used with |
| 28.69 | | good tablet | | any drug except the |
| Pearlitol 400DC: | | USP basket | | drugs that have |
| 37.06 | | rack assembly: | | amine group. |
| Advantose 100: 12.35 | | 12 s | | |
| Avicel PH 101: 15.0 | | | | |
| Kollidon CL: 2.0 | | | | |
| L-HPC 11: 2.0 | | | | |
| Citric acid: 1.0 | | | | |
| AsesulK: 0.2 | | | | |
| Syloid: 0.5 | | | | |
| Tangerine: 0.2 | | | | |
| Pruv: 1.0 | | | | |
| Lot#/mfg date: 1242- | | | | |
| 158/ | | | | |
| Feb. 6, 2002 | | | | |
| 250 g batch/11 mm Flat | | | | |
| Face Radial | | | | |
| Edge/450 mg | | | | |

### Fast Disintegrating Non Floss Tablet Additional Preferred Formulation

| | | | | |
|---|---|---|---|---|
| Fluoxetine TMMS: | 28.4 | Mouth: 8-10 s | 0.5 | Can be used with |
| 28.69 | | very good | | any drug |
| Pearlitol 400DC: | | tablet | | |
| 48.41 | | USP basket | | |
| Avicel PH 101: 16.0 | | rack assembly: | | |
| Kollidon CL: 2.0 | | 12 s | | |
| L-HPC 11: 2.0 | | | | |
| Citric acid: 1.0 | | | | |
| AsesulK: 0.2 | | | | |
| Magnasweet 100: 0.2 | | | | |
| Tangerine: 0.2 | | | | |
| Syloid: 0.5 | | | | |
| Pruv: 1.0 | | | | |
| Lot#/mfg date: 1242- | | | | |
| 167/ | | | | |
| Feb. 13, 2002 | | | | |
| 250 g batch/11 mm Flat | | | | |
| Face Radial | | | | |
| Edge/450 mg | | | | |

### Additional Formulations:

| | | Mixing procedure & | Hardness | Disintegration | |
|---|---|---|---|---|---|
| Formulation Lot# | Objective | Equipment used | (N) | Time | Friability % |
| Fluoxetine TMMS: | Investigate | ½ Pearlitol 400DC, | 32.4 | Mouth: 10 S | Abrasion: |
| 28.69 | high level | all MS | | | 0.3 |
| Pearlitol 400DC: | of Kollidon | ½ Pearlitol 400DC, | | | Drop: |
| 58.41 | XL for fast | mix for 3 min. Add | | | 2.1 |
| Kolidon XL: 10 | disintegration | all Citric acid, all | | | |
| Citric acid: 1.0 | using | AcesuK, all syloid, | | | |
| AsesulK: 0.2 | high | all Kollidon, all | | | |
| Tangerine: 0.2 | compression. | tangerine, mix for | | | |
| Syloid: 0.5 | | 5 min. Then pour all | | | |
| Pruv: 1.0 | | pruv and mix for 2 min | | | |
| Lot#/mfg date: | | using Turbula | | | |
| 1242-117/Jan. 14, 2002 | | mixer. | | | |
| 250 g batch | | Piccola tablets press | | | |
| | | 11 mm punch FFRE | | | |
| | | 450 mg table | | | |
| Fluoxetine TMMS: | Evaluate | ½ Parteck M200, all | 22.2 | Mouth: 10 S | Abrasion: |
| 28.69 | different | MS, | | | 1.4 |
| Pearlitol 400DC: | from | ½ Parteck M200 mix | | | Drop: |
| 58.41 | different | for 3 min. Add all | | | 4.1 |
| Kolidon XL: 10 | suppliers. | Citric acid, all | | | |
| Citric acid: 1.0 | | AcesuK, all syloid, | | | |
| AsesulK: 0.2 | | all Kollidon, all | | | |
| Tangerine: 0.2 | | tangerine, mix for 5 min. | | | |
| Syloid: 0.5 | | Then pour all | | | |
| Pruv: 1.0 | | pruv and mix for 2 min. | | | |
| Lot#/mfg date: | | using Turbula | | | |
| 1242-118/Jan. 14, 2002 | | mixer. | | | |
| 250 g batch | | Piccola tablets press | | | |
| | | 11 mm punch FFRE | | | |
| | | 450 mg tablet | | | |
| Fluoxetine TMMS: | Evaluate | ½ Parteck M300, all | 29.9 | Mouth: 10 S | Abrasion: |
| 28.69 | different | MS | | | 0.8 |
| Pearlitol 400DC: | mannitol | ½ Parteck M300, mix | | | Drop: |
| 58.41 | from | for 3 min. Add all | | | 3.0 |
| Kolidon XL: 10 | different | Citric acid, all | | | |
| Citric acid: 1.0 | suppliers. | AcesuK, all syloid, | | | |
| AsesulK: 0.2 | | all Kollidon, all | | | |
| Tangerine: 0.2 | | tangerine, mix for 5 min. | | | |
| Syloid: 0.5 | | Then pour all | | | |
| Pruv: 1.0 | | pruv and mix for 2 min. | | | |
| Lot#/mfg date: | | using Turbula | | | |
| 1242-119/Jan. 14, 2002 | | mixer. | | | |
| 250 g batch | | Piccola tablets press | | | |
| | | 11 mm punch FFRE | | | |
| | | 450 mg tablet | | | |
| Fluoxetine TMMS: | Increase | ½ Pearlitol 400DC, | 29.6 | Mouth: 10 S | Abrasion: |
| 28.69 | the | all MS | | | 0.4 |
| Pearlitol 400DC: | Kollidon | ½ pearlitol 400DC, | | | Drop: |
| 48.41 | XL from | mix for 3 min. Add | | | 2.3 |
| Kolidon XL: 20 | 10% to | all Citric acid, all | | | |
| Citric acid: 1.0 | 20% to | AcesuK, all syloid, | | | |
| AsesulK: 0.2 | determine | all Kollidon, all | | | |
| Tangerine: 0.2 | the effect | tangerine, mix for | | | |
| Syloid: 0.5 | of | 5 min. Then pour all | | | |
| Pruv: 1.0 | disintegrant | pruv and mix for 2 min | | | |
| Lot#/mfg date: | concentration | using Turbula | | | |
| 1242-120/Jan. 15, 2002 | on | mixer. | | | |
| 250 g batch | disintegration | Piccola tablets press | | | |
| | time | 11 mm punch FFRE | | | |
| Fluoxetine TMMS: | Investigate | ½ Pearlitol 400DC, | 16.2 | Mouth: 20 S, | Abrasion: |
| 28.69 | alternative | all MS | | at 20 and 30 | 14.8 |
| Pearlitol 400DC: | distintegrant | ½ Pearlitol 400DC, | | N tablets | Drop: |
| 48.41 | like L- | mix for 3 min. Add | | very slow to | Powder |
| L-HPC 11: 2.0 | HPC11 | all Citric acid, all | | disintegrate | collection |
| Citric acid: 1.0 | | AcesuK, all syloid, | | | |
| AsesulK: 0.2 | | all L-HPC, all | | | |
| Tangerine: 0.2 | | tangerine, mix for 5 min. | | | |
| Syloid: 0.5 | | Then pour all | | | |
| Pruv: 1.0 | | pruv and mix for 2 min | | | |
| Lot#/mfg date: | | using Turbula | | | |
| 1242-123/Jan. 16, 2002 | | mixer. | | | |
| 250 g batch | | Piccola tablets press | | | |
| | | 11 mm punch FFRE | | | |
| | | 450 mg tablet | | | |
| Fluoxetine TMMS: | Increase | ½ Pearlitol 400DC, | 29.7 | Mouth: 10 S | Abrasion: |
| 28.69 | the | all MS | | | 0.2 |
| Pearlitol 400DC: | Kollidon | ½ pearlitol 400DC, | | | Drop: |
| 48.41 | XL from | mix for 3 min. Add | | | 0.8 |
| Avicel PH 101: 16.0 | 10% to | all Citric acid, all | | | |
| L-HPC 11: 4.0 | 20% to | AcesuK, all syloid, | | | |
| Citric acid: 1.0 | determine | all avicel, all L-HPC, | | | |
| AsesulK: 0.2 | the effect | all tangerine, mix for | | | |
| Tangerine: 0.2 | of | 5 min. Then pour all | | | |
| Syloid: 0.5 | disintegrant | pruv and mix for 2 min | | | |
| Pruv: 1.0 | concentration | using Turbula | | | |
| Lot#/mfg date: | on | mixer. | | | |
| 1242-124/Jan. 16, 2002 | disintegration | Piccola tablets press | | | |
| 250 g batch | time | 11 mm punch FFRE | | | |
| Avicel PH101/L- | | 450 mg tablet | | | |
| HPC11 ratio | | | | | |
| (80/20) | | | | | |
| Fluoxetine TMMS: | Evaluate | ½ Pearl 400DC, all | 34.0 | Mouth: 10 S | Abrasion: |
| 28.69 | different | MS | | | 0.2 |
| Pearlitol 400DC: | ratio of | ½ Pearlitol 400DC, | | | Drop: |
| 48.41 | avicel PH | mix for 3 min. Add | | | 0.8 |
| Avicel PH 101: 18.0 | 101/L-HPC | all Citric acid, all | | | |
| L-HPC 11: 2.0 | 11 to | AcesuK, all syloid, | | | |
| Citric acid: 1.0 | determine | all avicel, all L0HPC, | | | |
| AsesulK: 0.2 | which | all tangerine, mix for | | | |
| Tangerine: 0.2 | excipient | 5 min. Then pour all | | | |
| Syloid: 0.5 | affect more | pruv and mix for 2 min | | | |
| Pruv: 1.0 | the | using Turbula | | | |
| Lot#/mfg date: | disintegration | mixer. | | | |
| 1242-125/Jan. 16, 2002 | in the | Piccola tablets press | | | |
| 250 g batch | mouth. | 11 mm punch FFRE | | | |
| Avicel PH 101/L- | | 450 mg table | | | |
| HPC11 ratio | | | | | |
| (90/10) | | | | | |
| Fluoxetine TMMS: | Evaluate | ½ Pearlitol 400DC, | 31.0 | Mouth: 10 S | Abrasion: |
| 28.69 | different | all MS, | | | 0.2 |
| Pearlitol 400DC: | ratio of | ½ Pearlitol 400DC, | | | Drop: |
| 48.41 | avicel PH | mix for 3 min. Add | | | 1.0 |
| Avicel PH 101: 18.0 | 101/L-HPC | all Citric acid, all | | | |
| L-HPC 11: 2.0 | 11 to | AcesuK, all syloid, | | | |
| Citric acid: 1.0 | determine | all Kollidon, all | | | |
| AsesulK: 0.2 | which | tangerine, mix for 5 min. | | | |
| Tangerine: 0.2 | excipient | Then pour all | | | |
| Syloid: 0.5 | affect more | pruv and mix for 2 min. | | | |
| Pruv: 1.0 | the | using Turbula | | | |
| Lot#/mfg date: | disintegration | mixer. | | | |
| 1242-129/Jan. 19, 2002 | in the | Piccola tablets press | | | |
| 250 g batch | mouth. | 11 mm punch FFRE | | | |
| Avicel PH 101/L- | | 450 mg tablet | | | |
| HPC11 ratio | | | | | |
| (90/10) | | | | | |
| Fluoxetine TMMS: | Comparative | ½ Pearlitol 400DC, | 33.8 | Mouth 10: | Abrasion: |
| 28.69 | study of | all MS, | | 10 S | 0.1 |
| Pearlitol 400DC: | disintegration | ½ Pearlitol 400DC, | | | Drop: |
| 48.41 | time of | mix for 3 min. Add | | | 1.5 |
| Avicel PH 101: 16.0 | avicel PH | all Citric acid, all | | | |
| Kollidon XL: 4.0 | 101/L- | Acesu K, all syloid, | | | |
| Citric acid: 1.0 | HPC11 | all avicel, all | | | |
| AsesulK: 0.2 | formulation | Kollidon, all | | | |
| Tangerine: 0.2 | versus | tangerine, mix for 5 min. | | | |
| Syloid: 0.5 | avicel PH | Then pour all | | | |
| Pruv: 1.0 | 101/Kollidon | pruv and mix for 2 min | | | |
| Lot#/mfg date: | XL | using Turbula | | | |
| 1242-126/Jan. 17, 2002 | | mixer. | | | |
| 250 g batch | | Piccola tablets press | | | |
| Avicel PH | | 11 mm punch FFRE | | | |
| 101/Kollidon ratio | | 450 mg tablet | | | |
| (80/20 | | | | | |
| Fluoxetine TMMS: | Comparative | ½ Pearlitol 400DC, | 31-37 | Mouth 10: | Abrasion: |
| 28.69 | study of | all MS, | | 10 S | 0.04 |
| Pearlitol 400DC: | disintegration | ½ Pearlitol 400DC, | | | Drop: |
| 48.41 | time of | mix for 3 min. Add | | | 1.6 |
| Avicel PH 101: 4.0 | avicel PH | all Citric acid, all | | | |
| Kollidon XL: 16.0 | 101/L- | Acesu K, all syloid, | | | |
| Citric acid: 1.0 | HPC11 | all avicel, all | | | |
| AsesulK: 0.2 | formulation | Kollidon, all | | | |
| Tangerine: 0.2 | versus | tangerine, mix for 5 min. | | | |
| Syloid: 0.5 | avicel PH | Then pour all | | | |
| Pruv: 1.0 | 101/Kollidon | pruv and mix for 2 min | | | |
| Lot#/mfg date: | XL | using Turbula | | | |
| 1242-127/Jan. 17, 2002 | | mixer. | | | |
| 250 g batch | | Piccola tablets press | | | |
| Avicel PH | | 11 mm punch FFRE | | | |
| 101/Kollidon ratio | | 450 mg tablet | | | |
| (20/80) | | | | | |
| Fluoxetine TMMS: | Comparative | ½ Pearlitol 400DC, | 36.4 | Mouth 10: | Abrasion: |
| 28.69 | study of | all MS, | | 10 S | 1.0 |
| Pearlitol 400DC: | disintegration | ½ Pearlitol 400DC, | | | Drop: |
| 52.41 | time of | mix for 3 min. Add | | | 2.5 |
| Koilidon XL: 16.0 | 16% | all Citric acid, all | | | |
| Citric acid: 1.0 | Kollidon to | Acesu K, all syloid, | | | |
| AsesulK: 0.2 | 10 and | all avicel, all | | | |
| Tangerine: 0.2 | 20% | Kollidon, all | | | |
| Syloid: 0.5 | | tangerine, mix for 5 min. | | | |
| Pruv: 1.0 | | Then pour all | | | |
| Lot#/mfg date: | | pruv and mix for 2 min | | | |
| 1242-130/Jan. 19, 2002 | | using Turbula | | | |
| 250 g batch | | mixer. | | | |
| | | Piccola tablets press | | | |
| | | 11 mm punch FFRE | | | |
| | | 450 mg tablet | | | |
| Fluoxetine TMMS: | Increase | ½ Pearlitol 400DC, | 29.4 | Mouth: 10 S | Abrasion: |
| 28.69 | the level of | all MS, | | | 1.7 |
| Pearlitol 400DC: | avicel to | ½ Pearlitol 400DC, | | | Drop: |
| 26.25 | improve | mix for 3 min. Add | | | 1.8 |
| Avicel PH 101: | the | all Citric acid, all | | | |
| 26.25 | disintegration | Acesu K, all syloid, | | | |
| L-HPC: 16 | time. | all avicel, all L-HPC, | | | |
| Citric acid: 1.0 | Avicel is | all tangerine, mix for | | | |
| AsesulK: 0.2 | porous and | 5 min. Then pour all | | | |
| Tangerine: 0.2 | therefore, it | pruv and mix for 2 min | | | |
| Syloid: 0.5 | absorbs lot | using Turbula | | | |
| Pruv: 1.0 | of water | mixer. | | | |
| Lot#/mfg date: | which | F tablets press | | | |
| 1242-131/Jan. 21, 2002 | helps the | 11 mm punch FFRE. | | | |
| 250 g batch | swelling of | 450 mg tablet | | | |
| | L-HPC | | | | |
| Fluoxetine TMMS: | Same | ½ Pearlitol 400DC, | 29.7 | Mouth: 10 S | Abrasion: |
| 28.69 | objective | all MS | | | 0.3 |
| Pearlitol 400DC: | as 1242- | ½ Pearlitol 400DC, | | | Drop: |
| 26.25 | 131, except | mix for 3 min. Add | | | 1.8 |
| Avicel PH 101: | Kollidon | all Citric acid, all | | | |
| 26.25 | was used. | AcesuK, all syloid, | | | |
| Kolidon XL: 16 | | all Kollidon, all | | | |
| Citric acid: 1.0 | | tangerine, mix for 5 min. | | | |
| AsesulK: 0.2 | | Then pour all | | | |
| Tangerine: 0.2 | | pruv and mix for 2 min | | | |
| Syloid: 0.5 | | using Turbula | | | |
| Pruv: 1.0 | | mixer. | | | |
| Lot#/mfg date: | | F tablets press | | | |
| 1242-132/Jan. 21, 2002 | | 11 mm punch FFRE | | | |
| 250 g batch | | 450 mg table | | | |
| Ireland Formulation | Enalapril | | 26 | Mouth: 10 S | Abrasion: |
| | FD tablets | | | | 2.5 |
| | 36 mg | | | | Drop: |
| | | | | | 0.3 |
| Fluoxetine TMMS: | Investigate | ½ Pearlitol 400DC, | 28.3 | Mouth: 15-20 S | Abrasion: |
| 28.69 | the effect | all MS | | Slower than | 0.3 |
| Pearlitol 400DC: | of MCC on | ½ Pearlitol 400DC, | | 1242-125 | Drop: |
| 54.41 | the | mix for 3 min. Add | | | 0.3 |
| Avicel PH 101: 12.0 | disintegration | all Citric acid, all | | | |
| L-HPC 11: 2.0 | of the | AcesuK, all syloid, | | | |
| Citric acid: 1.0 | tablets. | all avicel, all L- | | | |
| AsesulK: 0.2 | Decrease | HPCn, all tangerine, | | | |
| Tangerine: 0.2 | MCC from | mix for 5 min. Then | | | |
| Syloid: 0.5 | 18 to 12% | pour all pruv and mix | | | |
| Pruv: 1.0 | | for 2 min using | | | |
| Lot#/mfg date: | | Turbula mixer. | | | |
| 1242-133/Jan. 23, 2002 | | F tablets press | | | |
| 250 g batch | | 11 mm punch FFRE | | | |
| | | 450 mg table | | | |
| Fluoxetine TMMS: | Investigate | ½ Pearlitol 400DC, | 28.1 | Mouth: 20 S | Abrasion: |
| 28.69 | the effect | all MS | | Slower than | 0.4 |
| Pearlitol 400DC: | of MCC on | ½ Pearlitol 400DC, | | 1242-133 | Drop: |
| 60.41 | the | mix for 3 min. Add | | | 0.4 |
| Avicel PH 101: 6.0 | disintegration | all Citric acid, all | | | |
| L-HPC 11: 2.0 | of the | AcesuK, all syloid, | | | |
| Citric acid: 1.0 | tablets. | all avicel, all L-HPC, | | | |
| AsesulK: 0.2 | Decrease | all tangerine, mix for | | | |
| Tangerine: 0.2 | MCC from | 5 min. Then pour all | | | |
| Syloid: 0.5 | 18 to 6% | pruv and mix for 2 min | | | |
| Pruv: 1.0 | | using Turbula | | | |
| Lot#/mfg date: | | mixer. | | | |
| 1242-134/Jan. 23, 2002 | | F tablets press | | | |
| 250 g batch | | 11 mm punch FFRE | | | |
| | | 450 mg table | | | |
| Fluoxetine TMMS: | Decreasing | ½ Pearlitol 400DC, | 29.5 | Mouth: 10 S | Abrasion: |
| 28.69 | the level of | all MS | | As good as | 0.3 |
| Pearlitol 400DC: | MCC from | ½ Pearlitol 400DC, | | 1242-125 | Drop: |
| 51.41 | 18 to 12% | mix for 3 min. Add | | | 0.3 |
| Avicel PH 101: 15.0 | in the | all Citric acid, all | | | |
| L-HPC 11: 2.0 | formulation | AcesuK, all syloid, | | | |
| Citric acid: 1.0 | slowed | all avicel, all L-HPC, | | | |
| AsesulK: 0.2 | down | all tangerine, mix for | | | |
| Tangerine: 0.2 | slightly the | 5 min. Then pour all | | | |
| Syloid: 0.5 | disintegration | pruv and mix for 2 min | | | |
| Pruv: 1.0 | of the | using Turbula | | | |
| Lot#/mfg date: | tablets, but | mixer. | | | |
| 1242-135/Jan. 24, 2002 | it appeared | F tablets press | | | |
| 250 g batch | to be an | 11 mm punch FFRE | | | |
| | optimum | 450 mg table | | | |
| | level in | | | | |
| | between. | | | | |
| | The level | | | | |
| | of MCC | | | | |
| | was | | | | |
| | decreased | | | | |
| | to 15% | | | | |
| | instead. | | | | |
| Fluoxetine TMMS: | To | ½ Pearlitol 400DC, | 27.5 | Mouth: 20 S | Abrasion: |
| 28.69 | investigate | all MS | | Disintegrate | 0.2 |
| Pearlitol 400DC: | if the use | ½ Pearlitol 400DC, | | with a core | Drop: |
| 53.41 | of L-HPC | mix for 3 min. Add | | | 0.4 |
| Avicel PH 101: 15.0 | is | all Citric acid, all | | | |
| Citric acid: 1.0 | necessary | AcesuK, all syloid, | | | |
| AsesulK: 0.2 | in the | all avicel, all | | | |
| Tangerine: 0.2 | formulation | tangerine, mix for 5 min. | | | |
| Syloid: 0.5 | to enhance | Then pour all | | | |
| Pruv: 1.0 | the | pruv and mix for 2 min | | | |
| Lot#/mfg date: | disintegration | using Turbula | | | |
| 1242-136/Jan. 24, 2002 | of the | mixer. | | | |
| 250 g batch | tablet. | F tablets press | | | |
| | | 11 mm punch FFRE | | | |
| | | 450 mg table | | | |
| Fluoxetine TMMS: | Investigate | ½ Pearlitol 400DC, | 28.3 | Mouth: 20 S | Abrasion: |
| 28.69 | other | all MS | | As good as | 0.2 |
| Pearlitol 400DC: | grades of | ½ Pearlitol 400DC, | | 1242-125 | Drop: |
| 51.41 | MCC | mix for 3 min. Add | | | 0.4 |
| Prosolv 90: 15.0 | | all Citric acid, all | | | |
| L-HPC 11: 2.0 | | AcesuK, all syloid, | | | |
| Citric acid: 1.0 | | all prosolv, all | | | |
| AsesulK: 0.2 | | tangerine, mix for 5 min. | | | |
| Tangerine: 0.2 | | Then pour all | | | |
| Syloid: 0.5 | | pruv and mix for 2 min | | | |
| Pruv: 1.0 | | using Turbula | | | |
| Lot#/mfg date: | | mixer. | | | |
| 1242-137/Jan. 24, 2002 | | F tablets press | | | |
| 250 g batch | | 11 mm punch FFRE | | | |
| | | 450 mg table | | | |
| Fluoxetine TMMS: | Investigate | ½ Pearlitol 400DC, | 26.0 | Mouth: 10 S | Abrasion: |
| 28.69 | other | all MS | | Better than | 0.3 |
| Pearlitol 400DC: | grades of | ½ Pearlitol 400DC, | | 1242-124 | Drop: |
| 51.41 | MCC | mix for 3 min. Add | | | 0.2 |
| Prosolv 90: 15.0 | | all Citric acid, all | | | |
| L-HPC 11: 2.0 | | AcesuK, all syloid, | | | |
| Citric acid: 1.0 | | all prosolv, all L- | | | |
| AsesulK: 0.2 | | HPC, all tangerine, | | | |
| Tangerine: 0.2 | | mix for 5 min. Then | | | |
| Syloid: 0.5 | | pour all pruv and mix | | | |
| Pruv: 1.0 | | for 2 min using | | | |
| Lot#/mfg date: | | Turbula mixer. | | | |
| 1242-138/Jan. 24, 2002 | | F tablets press | | | |
| 250 g batch | | 11 mm punch FFRE | | | |
| | | 450 mg table | | | |
| Fluoxetine TMMS: | Investigate | ½ Pearlitol 400DC, | 28.4 | Mouth: 15 S-20 S | Abrasion: |
| 28.69 | other | all MS | | | 0.2 |
| Pearlitol 400DC: | greades of | ½ Pearlitol 400DC, | | | Drop: |
| 51.41 | MCC | mix for 3 min. Add | | | 0.2 |
| Avicel PH 102″ | | all Citric acid, all | | | |
| 15.0 | | AcesuK, all syloid, | | | |
| L-HPC 11: 2.0 | | all avicel, all L-HPC, | | | |
| Citric acid: 1.0 | | all tangerine, mix for | | | |
| AsesulK: 0.2 | | 5 min. Then pour all | | | |
| Tangerine: 0.2 | | pruv and mix for 2 min | | | |
| Syloid: 0.5 | | using Turbula | | | |
| Pruv: 1.0 | | mixer. | | | |
| Lot#/mfg date: | | F tablets press | | | |
| 1242-139/Jan. 24, 2002 | | 11 mm punch FFRE | | | |
| 250 g batch | | 450 mg table | | | |
| Fluoxetine TMMS: | Investigate | ½ Pearlitol 400DC, | 24.4 | Mouth: 15 S | Abrasion: |
| 28.69 | other | all MS | | | 0.3 |
| Pearlitol 400DC: | greades of | ½ Pearlitol 400DC, | | | Drop: |
| 53.41 | MCC | mix for 3 min. Add | | | 0.3 |
| Avicel PH 113: 15.0 | | all Citric acid, all | | | |
| L-HPC 11: 2.0 | | AcesuK, all syloid, | | | |
| Citric acid: 1.0 | | all avicel, all L-HPC, | | | |
| AsesulK: 0.2 | | all tangerine, mix for | | | |
| Tangerine: 0.2 | | 5 min. Then pour all | | | |
| Syloid: 0.5 | | pruv and mix for 2 min | | | |
| Pruv: 1.0 | | using Turbula | | | |
| Lot#/mfg date: | | mixer. | | | |
| 1242-140/Jan. 25, 2002 | | F tablets press | | | |
| 250 g batch | | 11 mm punch FFRE | | | |
| | | 450 mg table | | | |
| Fluoxetine TMMS: | To | ½ advantose, all MS | 26.9 | Mouth: 20 S | Abrasion: |
| 28.69 | investigate | ½ advantose, mix for | | with a core. | 0.8 |
| Advantose 100: | alternative | 3 min. Add all Citric | | Tablet sweet | Drop: |
| 68.41 | polyols. In | acid, all AcesuK, all | | and have | 2.0 |
| Citric acid: 1.0 | this | syloid, all tangerine, | | good | |
| AsesulK: 0.2 | experiment, | mix for 5 min. Then | | mouthfeel. | |
| Tangerine: 0.2 | determine | pour all pruv and mix | | | |
| Syloid: 0.5 | the | for 2 min using | | | |
| Pruv: 1.0 | compressibility | Turbula mixer. | | | |
| Lot#/mfg date: | of | F tablets press | | | |
| 1242-141/Jan. 25, 2002 | maltose | 11 mm punch FFRE | | | |
| 250 g batch | (advantose | 450 mg table | | | |
| | 100) | | | | |
| Fluoxetine TMMS: | To | ½ advantose, all MS | 27.9 | Mouth: 10 S | Abrasion: |
| 28.69 | investigate | ½ advantose, mix for | | Not as good | 1.0 |
| Advantose 100: | alternative | 3 min. Add all Citric | | as 1242-143 | Drop: |
| 53.41 | polyols. In | acid, all AcesuK, all | | | 4.2 |
| Prosolv 50: 15 | this | syloid, all Prosolv, all | | | |
| Citric acid: 1.0 | experiment, | tangerine, mix for 5 min. | | | |
| AsesulK: 0.2 | determine | Then pour all | | | |
| Tangerine: 0.2 | the | pruv and mix for 2 min | | | |
| Syloid: 0.5 | compressibility | using Turbula | | | |
| Pruv: 1.0 | of | mixer. | | | |
| Lot#/mfg date: | maltose | F tablets press | | | |
| 1242-142/Jan. 27, 2002 | (advantose | 11 mm punch FFRE | | | |
| 250 g batch | 100) and | 450 mg table | | | |
| | MCC | | | | |
| Fluoxetine TMMS: | To | ½ advantose, all MS | 27.9 | Mouth: 10 S | Abrasion: |
| 28.69 | investigate | ½ advantose, mix for | | Good tablets | 1.0 |
| Advantose 100: | alternative | 3 min. Add all Citric | | | Drop: |
| 51.41 | poyols. In | acid, all AcesuK, all | | | 3.7 |
| Prosolv 50: 15 | this | syloid, all Prosolv, all | | | |
| L-HPC 11: 2.0 | experiment, | tangerine, mix for 5 min. | | | |
| Citric acid: 1.0 | determine | Then pour all | | | |
| AsesulK: 0.2 | the | pruv and mix for 2 min | | | |
| Tangerine: 0.2 | compressibility | using Turbula | | | |
| Syloid: 0.5 | of | mixer. | | | |
| Pruv: 1.0 | maltose | F tablets press | | | |
| Lot#/mfg date: | (advantose | 11 mm punch FFRE | | | |
| 1242-143/Jan. 27, 2002 | 100)/ | 450 mg table | | | |
| 250 g batch | MCC/L- | | | | |
| | HPC | | | | |
| Fluoxetine TMMS: | To | ½ advantose, all MS | 26.3 | Mouth: 15 S | Abrasion: |
| 28.69 | investigate | ½ advantose, mix for | | Not as good | 0.6 |
| Advantose 100: | the effect of | 3 min. Add all Citric | | as 1242- | Drop: |
| 61.41 | MCC on the | acid, all AcesuK, all | | 143. | 1.8 |
| Prosolv 50: 5 | disintegration | syloid, all Prosolv, all | | | |
| L-HPC 11: 2.0 | of the | L-HPC, all tangerine, | | | |
| Citric acid: 1.0 | tablets | mix for 5 min. Then | | | |
| AsesulK: 0.2 | | pour all pruv and mix | | | |
| Tangerine: 0.2 | | for 2 min using | | | |
| Syloid: 0.5 | | Turbula mixer. | | | |
| Pruv: 1.0 | | F tablets press | | | |
| Lot#/mfg date: | | 11 mm punch FFRE | | | |
| 1242-144/Jan. 27, 2002 | | 450 mg table | | | |
| 250 g batch | | | | | |
| Fluoxetine TMMS: | To | ½ advantose, all MS | | Mouth: 20-25 S | Abrasion: |
| 28.69 | investigate | ½ advantose, mix for | | Not as good | 0.0 |
| Advantose 100: | the effect of | 3 min. Add all Citric | | as 1242- | Drop: |
| 56.41 | MCC on the | acid, all AcesuK, all | | 143. | 1.0 |
| Prosolv 50: 10.0 | disintegration | syloid, all Prosolv, all | | | |
| L-HPC 11: 2.0 | of the | L-HPC, all tangerine, | | | |
| Citric acid: 1.0 | tablets | mix for 5 min. Then | | | |
| AsesulK: 0.2 | | pour all pruv and mix | | | |
| Tangerine: 0.2 | | for 2 min using | | | |
| Syloid: 0.5 | | Turbula mixer. | | | |
| Pruv: 1.0 | | F tablets press | | | |
| Lot#/mfg date: | | 11 mm punch FFRE | | | |
| 1242-145/Jan. 27, 2002 | | 450 mg table | | | |
| 250 g batch | | | | | |
| Fluoxetine TMMS: | To compare | ½ advantose, all MS | 29.0 | Mouth: 10-15 S | Abrasion: |
| 28.69 | the use of | ½ advantose, mix for | | Good tablets | 1.0 |
| Advantose 100: | avicel to | 3 min. Add all Citric | | | Drop: |
| 51.41 | prosolv and | acid, all AcesuK, all | | | 2.0 |
| Avicel PH 101: | their effect | syloid, all syloid, all | | | |
| 15.0 | on friability | avicel, all tangerine, | | | |
| L-HPC 11: 2.0 | | mix for 5 min. Then | | | |
| Citric acid: 1.0 | | pour all pruv and mix | | | |
| AsesulK: 0.2 | | for 2 min using | | | |
| Tangerine: 0.2 | | Turbula mixer. | | | |
| Syloid: 0.5 | | F tablets press | | | |
| Pruv: 1.0 | | 11 mm punch FFRE | | | |
| Lot#/mfg date: | | 450 mg table | | | |
| 1242-146/Feb. 4, 2002 | | | | | |
| 250 g batch | | | | | |
| Fluoxetine TMMS: | To | ½ advantose, ½ | 27.8 | Mouth: 10 S | Abrasion: |
| 28.69 | investigate | Pearlitol, all MS, ½ | | Good tablets | 0.5 |
| Advantose 100: | the | Peqrlitol, ½ | | | Drop: |
| 25.70 | combination | advantose, mix for 3 min. | | | 1.9 |
| Pearlitol 400DC: | of 2 polyols | Add all Citric | | | |
| 25.71 | at different | acid, all Acesu K, all | | | |
| Avicel PH 101: | ratio and | syloid, all avicel, all | | | |
| 15.0 | their effect | L-HPC, all tangerine, | | | |
| L-HPC 11: 2.0 | on | mix for 5 min. Then | | | |
| Citric acid: 1.0 | disintegration | pour all pruv and mix | | | |
| AsesulK: 0.2 | and | for 2 min using | | | |
| Tangerine: 0.2 | friability. | Turbula mixer. | | | |
| Syloid: 0.5 | | F tablets press | | | |
| Pruv: 1.0 | | 11 mm punch FFRE | | | |
| Lot#/mfg date: | | 450 mg table | | | |
| 1242-147/Feb. 4, 2002 | | | | | |
| 250 g batch | | | | | |
| Advantose | | | | | |
| 100/Perlitol | | | | | |
| 400DC (50/50) | | | | | |
| ratio | | | | | |
| Fluoxetine TMMS: | To | ½ advantose, ½ | 28.4 | Mouth: 15 S | Abrasion: |
| 28.69 | investigate | Pearlitol, all MS, ½ | | Good tablets | 0.3 |
| Advantose 100: | the | Pearlitol, ½ | | No | Drop: |
| 12.85 | combination | advantose, mix for 3 min. | | significant | 0.5 |
| Pearlitol 400DC: | of 2 polyols | Add all Citric | | difference | |
| 38.56 | at different | acid, all Acesu K, all | | between | |
| Avicel PH 101: | ratio and | syloid, all avicel, all | | 1242-147 | |
| 15.0 | their effect | L-HPC, all tangerine, | | | |
| L-HPC 11: 2.0 | on | mix for 5 min. Then | | | |
| Citric acid: 1.0 | disintegration | pour all pruv and mix | | | |
| AsesulK: 0.2 | and | for 2 min using | | | |
| Tangerine: 0.2 | friability. | Turbula mixer. | | | |
| Syloid: 0.5 | | F tablets press | | | |
| Pruv: 1.0 | | 11 mm punch FFRE | | | |
| Lot#/mfg date: | | 450 mg table | | | |
| 1242-148/Feb. 4, 2002 | | | | | |
| 250 g batch | | | | | |
| Advantose | | | | | |
| 100/Perlitol | | | | | |
| 400DC (25/75) | | | | | |
| ratio | | | | | |
| Fluoxetine TMMS: | To | ½ advantose, ½ | 28.4 | Mouth: 10 S | Abrasion: |
| 28.69 | investigate | Pearlitol, all MS, ½ | | Good tablets | 0.5 |
| Advantose 100: | the | Pearlitol, ½ | | Faster than | Drop: |
| 38.56 | combination | advantose, mix for 3 min. | | 1242-147 & | 1.6 |
| Pearlitol 400DC: | of 2 polyols | Add all Citric | | 148 | |
| 12.85 | at different | acid, all Acesu K, all | | | |
| Avicel PH 101: | ratio and | syloid, all avicel, all | | | |
| 15.0 | their effect | L-HPC, all tangerine, | | | |
| L-HPC 11: 2.0 | on | mix for 5 min. Then | | | |
| Citric acid: 1.0 | disintegration | pour all pruv and mix | | | |
| AsesulK: 0.2 | and | for 2 min using | | | |
| Tangerine: 0.2 | friability. | Turbula mixer. | | | |
| Syloid: 0.5 | | F tablets press | | | |
| Pruv: 1.0 | | 11 mm punch FFRE | | | |
| Lot#/mfg date: | | 450 mg table | | | |
| 1242-149/Feb. 4, 2002 | | | | | |
| 250 g batch | | | | | |
| Advantose | | | | | |
| 100/Perlitol | | | | | |
| 400DC (75/25) | | | | | |
| ratio | | | | | |
| Fluoxetine TMMS: | To compare | ½ Pearlitol, all MS | 27.1 | Mouth: 35 S | Abrasion: |
| 28.69 | the physical | ½ Pearlitol, mix for 3 min. | | Very slow | 0.2 |
| Pearlitol 400DC: | properties of | Add all Citric | | | Drop: |
| 68.41 | pearlitol to | acid, all AcesuK, all | | | 0.3 |
| Citric acid: 1.0 | advantols | syloid, all syloid, all | | | |
| AsesulK: 0.2 | | tangerine, mix for 5 min. | | | |
| Tangerine: 0.2 | | Then pour all | | | |
| Syloid: 0.5 | | pruv and mix for 2 min | | | |
| Pruv: 1.0 | | using Turbula | | | |
| Lot#/mfg date: | | mixer. | | | |
| 1242-151/Feb. 4, 2002 | | F tablets press | | | |
| 250 g batch | | 11 mm punch FFRE | | | |
| | | 450 mg table | | | |
| Fluoxetine TMMS: | To evaluate | ½ Pearlitol, all MS | 33.9 | Mouth: 7-10 S | Abrasion: |
| 28.69 | the Kollidon | ½ Pearlitol, mix for 3 min. | | Very fast | 0.2 |
| Pearlitol 400DC: | CL and its | Add all Citric | | tablet | Drop: |
| 51.41 | effect on | acid, all AcesuK, all | | | 0.6 |
| Avicel PH 101: | disintegration | syloid, all syloid, all | | | |
| 15.0 | and | avicel, all kollidon, | | | |
| Kollidon CL: 2.0 | friability in | all tangerine, mix for | | | |
| Citric acid: 1.0 | the pearlitol | 5 min. Then pour all | | | |
| AsesulK: 0.2 | formulation. | pruv and mix for 2 min | | | |
| Tangerine: 0.2 | | using Turbula | | | |
| Syloid: 0.5 | | mixer. | | | |
| Pruv: 1.0 | | F tablets press | | | |
| Lot#/mfg date: | | 11 mm punch FFRE | | | |
| 1242-152/Feb. 5, 2002 | | 450 mg table | | | |
| 250 g batch | | | | | |
| Fluoxetine TMMS: | To evaluate | ½ advantose, ½ | 30.8 | Mouth: 10 S | Abrasion: |
| 28.69 | the Kollidon | Pearlitol, all MS, ½ | | Very fast | 0.2 |
| Pearlitol 400Dc: | CL and its | Pearlitol, ½ | | tablet no | Drop: |
| 38.56 | effect on | advantose, mix for 3 min. | | difference to | 0.2 |
| Advantose 100: | disintegration | Add all Citric | | 1242-152. | |
| 51.41 | and | acid, all AcesuK, all | | At 40N, | |
| Avicel PH 101: | friability in | syloid, all syloid, all | | tablets | |
| 15.0 | the pearlitol | avicel, all kollidon, | | disintegrate | |
| Kollidon CL: 2.0 | formulation. | all tangerine, mix for | | within 15 s. | |
| Citric acid: 1.0 | | 5 min. Then pour all | | | |
| AsesulK: 0.2 | | pruv and mix for 2 min | | | |
| Tangerine: 0.2 | | using Turbula | | | |
| Syloid: 0.5 | | mixer. | | | |
| Pruv: 1.0 | | F tablets press | | | |
| Lot#/mfg date: | | 11 mm punch FFRE | | | |
| 1242-153/Feb. 4, 2002 | | 450 mg table | | | |
| 250 g batch | | | | | |
| Fluoxetine TMMS: | Optimize | ½ Pearlitol, all MS | 35.7 | Mouth: 15 S | Abrasion: |
| 28.69 | the avicel | ½ Pearlitol, mix for 3 min. | | Not as fast | 0.2 |
| Pearlitol 400DC: | level | Add all Citric | | as 15% | Drop: |
| 56.41 | | acid, all AcesuK, all | | avicel | 0.3 |
| Avicel PH 101: | | syloid, all syloid, all | | | |
| 10.0 | | avicel, all kollidon, | | | |
| Kollidon CL: 2.0 | | all tangerine, mix for | | | |
| Citric acid: 1.0 | | 5 min. Then pour all | | | |
| AsesulK: 0.2 | | pruv and mix for 2 min | | | |
| Tangerine: 0.2 | | using Turbula | | | |
| Syloid: 0.5 | | mixer. | | | |
| Pruv: 1.0 | | F tablets press | | | |
| Lot#/mfg date: | | 11 mm punch FFRE | | | |
| 1242-154/Feb. 5, 2002 | | 450 mg table | | | |
| 250 g batch | | | | | |
| Fluoxetine TMMS: | Optimize | ½ advantose, ½ | 26.7 | Mouth: 10-15 S | Abrasion: |
| 28.69 | the avicel | Pearlitol, all MS ½ | | Not as fast | 0.3 |
| Pearlitol 400DC: | level | Pearlitol, ½ | | 15% avicel | Drop: |
| 42.31 | | advantose, mix for 3 min. | | | 0.8 |
| Advantose 100: | | Add all Citric | | | |
| 51.41 | | acid, all AcesuK, all | | | |
| Avicel PH 101: | | syloid, all syloid, all | | | |
| 15.0 | | avicel, all kollidon, | | | |
| Kollidon CL: 2.0 | | tangerine, mix for 5 min. | | | |
| Citric acid: 1.0 | | Then pour all | | | |
| AsesulK: 0.2 | | pruv and mix for 2 min | | | |
| Tangerine: 0.2 | | using Turbula | | | |
| Syloid: 0.5 | | mixer. | | | |
| Pruv: 1.0 | | F tablets press | | | |
| Lot#/mfg date: | | 11 mm punch FFRE | | | |
| 1242-155/Feb. 5, 2002 | | 450 mg table | | | |
| 250 g batch | | | | | |
| Fluoxetine TMMS: | Optimize | ½ advantose, ½ | 21.6 | Mouth: 35 S | Abrasion: |
| 28.69 | the level of | Pearlitol, all MS, ½ | | Very slow | 0.2 |
| Pearlitol 400DC: | avicel | Pearlitol, ½ | | | Drop: |
| 49.81 | | advantose mix for 3 min. | | | 0.3 |
| Advantose 100: | | Add all Citric | | | |
| 16.60 | | acid, all AcesuK, all | | | |
| Kollidon CL: 2.0 | | syloid, all syloid, all | | | |
| Citric acid: 1.0 | | kollidon, tangerine, | | | |
| AsesulK: 0.2 | | mix for 5 min. Then | | | |
| Tangerine: 0.2 | | pour all pruv and mix | | | |
| Syloid: 0.5 | | for 2 min using | | | |
| Pruv: 1.0 | | Turbula mixer. | | | |
| Lot#/mfg date: | | F tablets press | | | |
| 1242-156/Feb. 5, 2002 | | 11 mm punch FFRE | | | |
| 250 g batch | | 450 mg table | | | |
| Fluoxetine TMMS: | To evalute | ½ Pearlitol, all MS | 29.4 | Mouth: 10 S | Abrasion: |
| 28.69 | the | ½ Pearlitol, mix for 3 min. | | Very fast | 0.4 |
| Pearlitol 400DC: | combination | Add all Citric | | tablet, no | Drop: |
| 49.41 | of Kollidon | acid, all AcesuK, all | | difference | 0.6 |
| Avicel PH 101: | CL/L0HPC | syloid, all syloid, all | | between | |
| 15.0 | and their | kollidon, all-HPC, | | 1242-154 & | |
| Kollidon CL: 2.0 | synergetic | all tangerine, mix for | | 140 batches | |
| L-HPC 11: 2.0 | effect on | 5 min. Then pour all | | | |
| Citric acid: 1.0 | disintegration | pruv and mix for 2 min | | | |
| AsesulK: 0.2 | and | using Turbula | | | |
| Tangerine: 0.2 | friability | mixer. | | | |
| Syloid: 0.5 | formulation. | F tablets press | | | |
| Pruv: 1.0 | | 11 mm punch FFRE | | | |
| Lot#/mfg date: | | 450 mg table | | | |
| 1242-157/Feb. 6, 2002 | | | | | |
| 250 g batch | | | | | |
| Fluoxetine TMMS: | To evalute | ½ advantose, ½ | 33.1 | Mouth: 12-15 S | Abrasion: |
| 28.69 | the | Pearlitol, all MS, ½ | | Good tablets | 0.3 |
| Pearlitol 400DC: | combination | Pearlitol, ½ | | | Drop: |
| 37.06 | of Kollidon | advantose, mix for 3 min. | | | 0.6 |
| Advantose 100: | CL/L0HPC | Add all Citric | | | |
| 12.35 | and their | acid, all AcesuK, all | | | |
| Avicel PH 101: | synergetic | syloid, all syloid, all | | | |
| 15.0 | effect on | kollidon, all | | | |
| Kollidon CL: 2.0 | disintegration | tangerine, mix for 5 min. | | | |
| L-HPC 11: 2.0 | and | Then pour all | | | |
| Citric acid: 1.0 | friability | pruv and mix for 2 min | | | |
| AsesulK: 0.2 | formulation. | using Turbula | | | |
| Tangerine: 0.2 | | mixer. | | | |
| Syloid: 0.5 | | F tablets press | | | |
| Pruv: 1.0 | | 11 mm punch FFRE | | | |
| Lot#/mfg date: | | 450 mg table | | | |
| 1242-158/Feb. 6, 2002 | | | | | |
| 250 g batch | | | | | |
| Fluoxetine TMMS: | To evaluate | ½ lab, all MS ½ lab, | 25.3 | Mouth: 10 S | Abrasion: |
| 28.69 | alternative | mix for 3 min. Add | | Good tablets | 0.6 |
| Lab 3038: 51.41 | polyols with | all Citric acid, all | | | Drop: |
| Avicel PH 101: | Kollidon | AcesuK, all syloid, | | | 2.0 |
| 15.0 | and their | all syloid, all | | | |
| Kollidon CL: 2.0 | effect on | kollidon, all | | | |
| Citric acid: 1.0 | disintegration | tangerine, mix for 5 min. | | | |
| AsesulK: 0.2 | | Then pour all | | | |
| Tangerine: 0.2 | | pruv and mix for 2 min | | | |
| Syloid: 0.5 | | using Turbula | | | |
| Pruv: 1.0 | | mixer. | | | |
| Lot#/mfg date: | | F tablets press | | | |
| 1242-159/Feb. 6, 2002 | | 11 mm punch FFRE | | | |
| 250 g batch | | 450 mg table | | | |
| Fluoxetine TMMS: | To evaluate | ½ lab, all MS ½ lab, | 32.4 | Mouth: 20 S | Abrasion: |
| 28.69 | alternative | mix for 3 min. Add | | | 0.2 |
| Lab 3038: 68.41 | polyols with | all Citric acid, all | | | Drop: |
| Avicel PH 101: | L-HPC and | AcesuK, all syloid, | | | 0.8 |
| 15.0 | their effect | all syloid, all HPC, | | | |
| L-HPC 11: 2.0 | on | all tangerine, mix for | | | |
| Citric acid: 1.0 | disintegration. | 5 min. Then pour all | | | |
| AsesulK: 0.2 | | pruv and mix for 2 min | | | |
| Tangerine: 0.2 | | using Turbula | | | |
| Syloid: 0.5 | | mixer. | | | |
| Pruv: 1.0 | | F tablets press | | | |
| Lot#/mfg date: | | 11 mm punch FFRE | | | |
| 1242-160/Feb. 6, 2002 | | 450 mg table | | | |
| 250 g batch | | | | | |

### Additional Non-Floss Formulations

| | | Mixing | | | | |
|---|---|---|---|---|---|---|
| | | procedure & | | | | |
| | | Equipment | Hardness | Disintegration | | |
| Formulation Lot# | Objective | Used | (N) | time | Friability % | Dissolution % |
| Fluoxetine TMMS: | Investigate | ½ Pearl | 32 | Mouth: 10 S | Abrasion: | |
| 28.69 | high | 400DC, all | | | 0.3 | |
| Pearlitol 400DC: | level of | MS½ pearlitol | | | Drop: | |
| 58.41 | Kollidon | 400DC, mix | | | 2.1 | |
| Kolidon XL: 10 | XL for | for 3 min. Add | | | | |
| Citric acid: 1.0 | fast | all Citric acid, | | | | |
| AsesulK: 0.2 | disintegration | all AcesuK, all | | | | |
| Syloid: 0.5 | using | syloid, all | | | | |
| Tangerine: 0.2 | high | Kollidon, all | | | | |
| Pruv: 1.0 | compression. | tangerine, mix | | | | |
| Lot# 1242-117 | | for 5 min. Then | | | | |
| | | pour all pruv | | | | |
| | | and mix for 2 min | | | | |
| | | using | | | | |
| | | Turbula mixer. | | | | |
| | | Piccola tablets | | | | |
| | | press | | | | |
| | | 11 mm punch | | | | |
| | | FFRE | | | | |
| Fluoxetine TMMS: | Evaluate | ½ Parteck | 22.2 | Mouth: 10 S | Abrasion: | |
| 28.69 | different | M200, all MS, | | | 1.4 | |
| Parteck M200: | mannitol | ½ Parteck | | | Drop: | |
| 58.41 | from | M200 mix for | | | 4.1 | |
| Kolidon XL: 10 | different | 3 min. Add all | | | | |
| Citric acid: 1.0 | suppliers. | Citric acid, all | | | | |
| AsesulK: 0.2 | | AcesuK, all | | | | |
| Syloid: 0.5 | | syloid, all | | | | |
| Tangerine: 0.2 | | Kollidon, all | | | | |
| Pruv: 1.0 | | tangerine, mix | | | | |
| Lot# 1242-118 | | for 5 min. | | | | |
| | | Then pour all | | | | |
| | | pruv and mix | | | | |
| | | for 2 min. | | | | |
| | | using Turbula | | | | |
| | | mixer. | | | | |
| | | Piccola tablets | | | | |
| | | press | | | | |
| | | 11 mm punch | | | | |
| | | FFRE | | | | |
| Fluoxetine TMMS: | Evaluate | ½ Parteck | 30.0 | Mouth: 10 S | Abrasion: | |
| 28.69 | different | M300, all MS, | | | 0.8 | |
| Parteck M300: | mannitol | ½ Parteck | | | Drop: | |
| 58.41 | from | M300, mix for | | | 3.0 | |
| Kolidon XL: 10 | different | 3 min. Add all | | | | |
| Citric acid: 1.0 | suppliers. | Citric acid, all | | | | |
| AsesulK: 0.2 | | Acesu K, all | | | | |
| Syloid: 0.5 | | syloid, all | | | | |
| Tangerine: 0.2 | | Kollidon, all | | | | |
| Pruv: 1.0 | | tangerine, mix | | | | |
| Lot# 1242-119 | | for 5 min. | | | | |
| | | Then pour all | | | | |
| | | pruv and mix | | | | |
| | | for 2 min. | | | | |
| | | using Turbula | | | | |
| | | mixer. | | | | |
| | | Piccola tablets | | | | |
| | | press | | | | |
| | | 11 mm punch | | | | |
| | | FFRE | | | | |
| Fluoxetine TMMS: | Increase | ½ Pearlitol | 27.0 | Mouth: 10 S | Abrasion: | |
| 28.69 | the | 400DC, all | | | 0.4 | |
| Pearlitol 400DC: | Kollidon | MS, | | | Drop: | |
| 48.41 | XL from | ½ Pearlitol | | | 2.3 | |
| Kolidon XL: 20 | 10% to | 400DC, mix | | | | |
| Citric acid: 1.0 | 20% to | for 3 min. Add | | | | |
| AsesulK: 0.2 | determine | all Citric acid, | | | | |
| Syloid: 0.5 | the | all Acesu K, all | | | | |
| Tangerine: 0.2 | effect of | syloid, all | | | | |
| Pruv: 1.0 | disintegrant | Kollidon, all | | | | |
| Lot# 1242-120 | concentration | tangerine, mix | | | | |
| | on | for 5 min. | | | | |
| | disintegration. | Then pour all | | | | |
| | time | pruv and mix | | | | |
| | | for 2 min. | | | | |
| | | using Turbula | | | | |
| | | mixer. | | | | |
| | | Piccola tablets | | | | |
| | | press | | | | |
| | | 11 mm punch | | | | |
| | | FFRE | | | | |
| Fluoxetine TMMS: | Investigate | ½ Pearlitol | 16.2 | Mouth: | Abrasion: | |
| 28.69 | alternative | 400DC, all | | 20 S, at 20 | 14.8 | |
| Pearlitol 400DC: | disintegrant | MS, | | and 30 N | Drop: | |
| 48.41 | like L- | ½ Pearlitol | | tablets very | powder | |
| L-HPC11: 20 | HPC11 | 400DC, mix | | slow to | collection | |
| Citric acid: 1.0 | | for 3 min. Add | | disintegrate | | |
| AsesulK: 0.2 | | all Citric acid, | | | | |
| Syloid: 0.5 | | all Acesu K, all | | | | |
| Tangerine: 0.2 | | syloid, all L- | | | | |
| Pruv: 1.0 | | HPC, all | | | | |
| Lot# 1242-123 | | tangerine, mix | | | | |
| | | for 5 min. | | | | |
| | | Then pour all | | | | |
| | | pruv and mix | | | | |
| | | for 2 min. | | | | |
| | | using Turbula | | | | |
| | | mixer. | | | | |
| | | Piccola tablets | | | | |
| | | press | | | | |
| | | 11 mm punch | | | | |
| | | FFRE | | | | |
| Fluoxetine TMMS: | Introduce | ½ Pearlitol | 30.0 | Mouth: 10 S | Abrasion: | |
| 28.69 | microcry | 400DC, all | | | 0.2 | |
| Pearlitol 400DC: | stalline | MS, | | | Drop: | |
| 48.41 | cellulose | ½ Pearlitol | | | 0.8 | |
| Avicel PH 101: 16.0 | as a | 400DC, mix | | | | |
| L-HPC 11: 4.0 | wicking | for 3 min. Add | | | | |
| Citric acid: 1.0 | and | all Citric acid, | | | | |
| AsesulK: 0.2 | dispersing | all Acesu K, all | | | | |
| Syloid: 0.5 | agent to | syloid, all | | | | |
| Tangerine: 0.2 | improve | avicel, all L- | | | | |
| Pruv: 1.0 | the | HPC, all | | | | |
| Lot# 1242-124 | disintegration | tangerine, mix | | | | |
| Avicel PH101/L- | of | for 5 min.. | | | | |
| HPC11 ratio (80/20) | the | Then pour all | | | | |
| | tablets. | pruv and mix | | | | |
| | | for 2 min using | | | | |
| | | Turbula mixer. | | | | |
| | | Piccola tablets | | | | |
| | | press | | | | |
| | | 11 mm punch | | | | |
| | | FFRE | | | | |
| Fluoxetine TMMS: | Evaluate | ½ Pearlitol | 34.0 | Mouth: 10 S | Abrasion: | |
| 28.69 | different | 400DC, all | | | 0.2 | |
| Pearlitol 400DC: | ratio of | MS, | | | Drop: | |
| 48.41 | avicel PH | ½ Pearlitol | | | 0.8 | |
| Avicel PH 101: 18.0 | 101/L- | 400DC, mix | | | | |
| L-HPC 11: 20 | HPC 11 | for 3 min. Add | | | | |
| Citric acid: 1.0 | to | all Citric acid, | | | | |
| AsesulK: 0.2 | determine | all Acesu K, all | | | | |
| Syloid: 0.5 | which | syloid, all | | | | |
| Tangerine: 0.2 | excipient | avicel, all L- | | | | |
| Pruv: 1.0 | affect | HPC, all | | | | |
| Lot# 1242-125 | more the | tangerine, mix | | | | |
| Avicel PH 101/L- | disintegration | for 5 min. | | | | |
| HPC11 ratio (90/10) | in | Then pour all | | | | |
| | the | pruv and mix | | | | |
| | mouth | for 2 min using | | | | |
| | | Turbula mixer | | | | |
| | | Piccola tablets | | | | |
| | | press | | | | |
| | | 11 mm punch | | | | |
| | | FFRE | | | | |
| Fluoxetine TMMS: | Evaluate | ½ Pearlitol | 34.0 | Mouth: 10 S | Abrasion: | |
| 28.69 | different | 400DC, all | | | 0.2 | |
| Pearlitol 400DC: | ratio of | MS, | | | Drop: | |
| 48.41 | avicel PH | ½ Pearlitol | | | 1.0 | |
| Avicel PH 101: 14.0 | 101/L- | 400DC, mix | | | | |
| L-HPC 11: 6.0 | HPC 11 | for 3 min. Add | | | | |
| Citric acid: 1.0 | to | all Citric acid, | | | | |
| AsesulK: 0.2 | determine | all Acesu K, all | | | | |
| Syloid: 0.5 | which | syloid, all | | | | |
| Tangerine: 0.2 | excipient | avicel, all L- | | | | |
| Pruv: 1.0 | affect | HPC, all | | | | |
| Lot# 1242-129 | more the | tangerine, mix | | | | |
| Avicel PH 101/L- | disintegration | for 5 min. | | | | |
| HPC11 ratio (70/30) | in | Then pour all | | | | |
| | the | pruv and mix | | | | |
| | mouth | for 2 min using | | | | |
| | | Turbula mixer. | | | | |
| | | Piccola tablets | | | | |
| | | press | | | | |
| | | 11 mm punch | | | | |
| | | FFRE | | | | |
| Fluoxetine TMMS: | Comparative | ½ Pearlitol | 34.0 | Mouth: 10 S | Abrasion: | |
| 28.69 | study | 400DC, all | | | 0.1 | |
| Pearlitol 400DC: | of | MS, | | | Drop: | |
| 48.41 | disintegration | ½ Pearlitol | | | 1.5 | |
| Avicel PH 101: 16.0 | time | 400DC, mix | | | | |
| Kollidon XL: 4.0 | of avicel | for 3 min. Add | | | | |
| Citric acid: 1.0 | PH | all Citric acid, | | | | |
| AsesulK: 0.2 | 101/L- | all Acesu K, all | | | | |
| Syloid: 0.5 | HPC11 | syloid, all | | | | |
| Tangerine: 0.2 | formulation | avicel, all | | | | |
| Pruv: 1.0 | versus | Killidon, all | | | | |
| Lot# 1242-126 | avicel PH | tangerine, mix | | | | |
| Avicel PH | 101/Kollidon | for 5 min. | | | | |
| 101/Kollidon ratio | XL | Then pour all | | | | |
| (80/20) | | pruv and mix | | | | |
| | | for 2 min using | | | | |
| | | Turbula mixer. | | | | |
| | | Piccola tablets | | | | |
| | | press | | | | |
| | | 11 mm punch | | | | |
| | | FFRE | | | | |
| Fluoxetine TMMS: | Comparative | ½ Pearlitol | 31-37 | Mouth: 10 S | Abrasion: | |
| 28.69 | study | 400DC, all | | | 0.04 | |
| Pearlitol 400DC: | of | MS, | | | Drop: | |
| 48.41 | disintegration | ½ Pearlitol | | | 1.6 | |
| Avicel PH 101: 4.0 | time | 400DC, mix | | | | |
| Kollidon XL: 16.0 | of avicel | for 3 min. Add | | | | |
| Citric acid: 1.0 | PH | all Citric acid, | | | | |
| AsesulK: 0.2 | 101/L- | all Acesu K, all | | | | |
| Syloid: 0.5 | HPC11 | syloid, all | | | | |
| Tangerine: 0.2 | formulation | avicel, all | | | | |
| Pruv: 1.0 | versus | Kollidon, all | | | | |
| Lot# 1242-127 | avicel PH | tangerine, mix | | | | |
| Avicel PH | 101/Kollidon | for 5 min. | | | | |
| 101/Kollidon ratio | | Then pour all | | | | |
| (20/80) | | pruv and mix | | | | |
| | | for 2 min using | | | | |
| | | Turbula mixer. | | | | |
| | | Piccola tablets | | | | |
| | | press | | | | |
| | | 11 mm punch | | | | |
| | | FFRE | | | | |
| Fluoxetine TMMS: | Comparative | ½ Pearlitol | 33.3 | Mouth: 10 S | Abrasion: | |
| 28.69 | study | 400DC, all | | | 1.0 | |
| Pearlitol 400DC: | of | MS, | | | Drop: | |
| 52.41 | disintegration | ½ Pearlitol | | | 2.5 | |
| Kollidon XL: 16.0 | time | 400DC, mix | | | | |
| Citric acid: 1.0 | of 16% | for 3 min. Add | | | | |
| AsesulK: 0.2 | Kollidon | all Citric acid, | | | | |
| Syloid: 0.5 | to 10 and | all Acesu K, all | | | | |
| Tangerine: 0.2 | 20% | syloid, all | | | | |
| Pruv: 1.0 | | Kollidon, all | | | | |
| Lot# 1242-130 | | tangerine, mix | | | | |
| | | for 5 min. | | | | |
| | | Then pour all | | | | |
| | | pruv and mix | | | | |
| | | for 2 min using | | | | |
| | | Turbula mixer. | | | | |
| | | Piccola tablets | | | | |
| | | press | | | | |
| | | 11 mm punch | | | | |
| | | FFRE. | | | | |
| Fluoxetine TMMS: | Increase | ½ Pearlitol | 29.4 | Mouth: 10 S | Abrasion: | |
| 28.69 | the level | 400DC, all | | | 1.7 | |
| Pearlitol 400DC: | of avicel | MS, | | | Drop: | |
| 26.25 | to | ½ Pearlitol | | | 1.8 | |
| Avicel PH 101: 26.25 | improve | 400DC, mix | | | | |
| L-HPC: 16 | the | for 3 min. Add | | | | |
| Citric acid: 1.0 | disintegration | all Citric acid, | | | | |
| AsesulK: 0.2 | time. | all Acesu K, all | | | | |
| Syloid: 0.5 | Avicel is | syloid, all | | | | |
| Tangerine: 0.2 | porous | avicel, all L- | | | | |
| Pruv: 1.0 | and | HPC, all | | | | |
| Lot# 1242-131 | therefore, | tangerine, mix | | | | |
| | it absorbs | for 5 min. | | | | |
| | lot of | Then pour all | | | | |
| | water | pruv and mix | | | | |
| | which | for 2 min | | | | |
| | helps the | using Turbula | | | | |
| | swelling | mixer. | | | | |
| | of L-HPC | F tablets press | | | | |
| | | 11 mm punch | | | | |
| | | FFRE. | | | | |
| Fluoxetine TMMS: | Same | ½ Pearlitol | 29.7 | Mouth: 10 S | Abrasion: | |
| 28.69 | objective as | 400DC, all | | | 0.3 | |
| Pearlitol 400DC: | 1242- | MS, | | | Drop: | |
| 26.25 | 131, except | ½ Pearlitol | | | 0.8 | |
| Avicel PH 101: 26.25 | Kollidon | 400DC, mix | | | | |
| Kolidon XL: 16 | was used. | for 3 min. Add | | | | |
| Citric acid: 1.0 | | all Citric acid, | | | | |
| AsesulK: 0.2 | | all Acesu K, all | | | | |
| Syloid: 0.5 | | syloid, all | | | | |
| Tangerine: 0.2 | | avicel, all | | | | |
| Pruv: 1.0 | | Kollidon, all | | | | |
| Lot# 1242-132 | | tangerine, mix | | | | |
| | | for 5 min. | | | | |
| | | Then pour all | | | | |
| | | pruv and mix | | | | |
| | | for 2 min. | | | | |
| | | using Turbula | | | | |
| | | mixer. | | | | |
| | | F tablets press | | | | |
| | | 11 mm punch | | | | |
| | | FFRE. | | | | |
| Ireland Formulation | Enapril | | 26 | Mouth: 10 S | Abrasion: | |
| EXP 988 | FD | | | | 2.5 | |
| | tablets | | | | Drop: | |
| | 36 mg | | | | 13.5 | |
| Fluoxetine TMMS: | Study the | ½ Pearlitol | 28.3 | Mouth: 15 | Abrasion: | |
| 28.69 | effect of | 400DC, all | | to 20 S | 0.3 | |
| Pearlitol 400DC: | avicel on | MS, | | | Drop: | |
| 54.41 | the | ½ Pearlitol | | | 0.3 | |
| Avicel PH 101: 12 | tablets | 400DC, mix | | | | |
| L-HPC: 2 | formulation | for 3 min. Add | | | | |
| Citric acid: 1.0 | at | all Citric acid, | | | | |
| AsesulK: 0.2 | differents | all Acesu K, all | | | | |
| Syloid: 0.5 | level | syloid, all | | | | |
| Tangerine: 0.2 | 12% and | avicel, all L- | | | | |
| Pruv: 1.0 | 6% as | HPC, all | | | | |
| Lot# 1242-133 | results of | tangerine, mix | | | | |
| | lot 1242- | for 5 min. | | | | |
| | 125 | Then pour all | | | | |
| | | pruv and mix | | | | |
| | | for 2 min | | | | |
| | | using Turbula | | | | |
| | | mixer. | | | | |
| | | F tablets press | | | | |
| | | 11 mm punch | | | | |
| | | FFRE. | | | | |
| Fluoxetine TMMS: | To | ½ Pearlitol | 28.1 | Mouth: 20 S | Abrasion: | |
| 28.69 | improve | 400DC, all | | slow | 0.4 | |
| Pearlitol 400DC: | the | MS, | | compared to | Drop: | |
| 60.41 | mouth | ½ Pearlitol | | 1242-133 | 0.4 | |
| Avicel PH 101: 6 | feel and | 400DC, mix | | | | |
| L-HPC: 2 | gritty | for 3 min. Add | | | | |
| Citric acid: 1.0 | taste of | all Citric acid, | | | | |
| AsesulK: 0.2 | the | all Acesu K, all | | | | |
| Syloid: 0.5 | tablets. | syloid, all | | | | |
| Tangerine: 0.2 | Avicel | avicel, all L- | | | | |
| Pruv: 1.0 | was | HPC, all | | | | |
| Lot# 1242-134 | reduced | tangerine, mix | | | | |
| | from | for 5 min. | | | | |
| | 18% to | Then pour all | | | | |
| | 12% by | pruv and mix | | | | |
| | keeping | for 2 min | | | | |
| | L-HPC | using Turbula | | | | |
| | 11 to 2% | mixer. | | | | |
| | level in | F tablets press | | | | |
| | tablets | 11 mm punch | | | | |
| | formulation | FFRE. | | | | |
| Fluoxetine TMMS: | As results | ½ Pearlitol | 29.5 | Mouth: 10 S | Abrasion: | |
| 28.69 | of | 400DC, all MS, | | | 0.3 | |
| Pearlitol 400DC: | 1242- | ½ Pearlitol | | | Drop: | |
| 51.41 | 125 and | 400DC, mix for | | | 0.3 | |
| Avicel PH 101: 15 | 1242-133 | 3 min. Add all | | | | |
| L-HPC: 2 | on the | Citric acid, all | | | | |
| Citric acid: 1.0 | tablets | Acesu K, all | | | | |
| AsesulK: 0.2 | disintegration, | syloid, all | | | | |
| Syloid: 0.5 | is | avicel, all L- | | | | |
| Tangerine: 0.2 | been | HPC, all | | | | |
| Pruv: 1.0 | found that | tangerine, mix | | | | |
| Lot# 1242-135 | the lot | for 5 min. Then | | | | |
| | 1242-125 | pour all pruv | | | | |
| | gave | and mix for 2 min | | | | |
| | better | using | | | | |
| | disintegration | Turbula mixer. | | | | |
| | which | F tablets press | | | | |
| | the level | 11 mm punch | | | | |
| | of Avicel | FFRE. | | | | |
| | was | | | | | |
| | increased | | | | | |
| | to 15% | | | | | |
| Fluoxetine TMMS: | Evaluate | ½ Pearlitol | 27.5 | Mouth: 20 S | Abrasion: | |
| 28.69 | the used | 400DC, all MS, | | | 0.2 | |
| Pearlitol 400DC: | of avicel | ½ Pearlitol | | | Drop: | |
| 53.41 | alone in | 400DC, mix for | | | 0.4 | |
| Avicel PH 101: 15 | the tablets | 3 min. Add all | | | | |
| Citric acid: 1.0 | formulation. | Citric acid, all | | | | |
| AsesulK: 0.2 | To | Acesu K, all | | | | |
| Syloid: 0.5 | determine | syloid, | | | | |
| Tangerine: 0.2 | the effect | all avicel, all | | | | |
| Pruv: 1.0 | of the | tangerine, mix | | | | |
| Lot# 1242-136 | disintegration | for 5 min. Then | | | | |
| | while | pour all pruv | | | | |
| | L-HPC11 | and mix for 2 min | | | | |
| | was | using | | | | |
| | removed. | Turbula mixer. | | | | |
| | | F tablets press | | | | |
| | | 11 mm punch | | | | |
| | | FFRE. | | | | |
| Fluoxetine TMMS: | Investigate | ½ Pearlitol | 28.3 | Mouth: 10 S | Abrasion: | |
| 28.69 | another | 400DC, all MS, | | better disint | 0.2 | |
| Pearlitol 400DC: | disintegrant | ½ Pearlitol | | than 1242- | Drop: | |
| 51.41 | Prosolv90 | 400DC, mix for | | 125 | 0.4 | |
| Prosolv90: 15 | to study | 3 min. Add all | | | | |
| L_HPC11: 2 | the | Citric acid, all | | | | |
| Citric acid: 1.0 | disintegration | Acesu K, all | | | | |
| AsesulK: 0.2 | properties | syloid, | | | | |
| Syloid: 0.5 | and | all Prosolv90, | | | | |
| Tangerine: 0.2 | compare | all L_HPC11, | | | | |
| Pruv: 1.0 | its | all tangerine, | | | | |
| Lot# 1242-137 | effectiveness | mix for 5 min. | | | | |
| | with | Then pour all | | | | |
| | avicel in a | pruv and mix for | | | | |
| | direct | 2 min using | | | | |
| | compaction. | Turbula mixer. | | | | |
| | | F tablets press | | | | |
| | | 11 mm punch | | | | |
| | | FFRE. | | | | |

Preferred Formulations Based on Directly Compressible Inorganic Salts, Alone or in Combination with a Cellulose Derivative:

The present preferred illustrative embodiments of the invention relate to the introduction of directly compressible inorganic salt with a cellulose derivative.

### Formulation I:

This formulation is based on an excipient mass containing a mixture of dibasic calcium phosphate dihydrate (Emcompress) and microcrystalline cellulose (Avicel).

| | % |
|---|---|
| Fluoxetine TMMS*: | 28.69 |
| Pearlitol 400DC | 36.31 |
| Emcompress: | 12.10 |
| Avicel PH 101: | 15.00 |
| L-HPC LH-11: | 2.00 |
| XL Kollidon: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation II:

This formulation is based on an excipient mass wherein mannitol is substituted with the dicalcium phosphate dihydrate.

| | % |
|---|---|
| Fluoxetine TMMS*: | 28.69 |
| Emcompress: | 48.41 |
| Avicel PH 101: | 15.00 |
| XL Kollidon: | 2.00 |
| L-HPC LH-11: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation III:

This formulation is based on an excipient mass wherein microcrystalline cellulose (Avicel) is substituted with the dicalcium phosphate dihydrate (Emcompress)

| | % |
|---|---|
| Fluoxetine TMMS*: | 28.69 |
| Pearlitol 400DC: | 48.41 |
| Emcompress: | 15.00 |
| L-HPC LH-11: | 2.00 |
| XL Kollidon: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation IV:

This formulation is based on an excipient mass containing a combination of Pearlitol 400DC/dicalcium phosphate dihydrate at ratio 75/25

| | % |
|---|---|
| Fluoxetine TMMS*: | 28.69 |
| Pearlitol 400DC: | 36.69 |
| Emcompress: | 12.10 |
| Avicel PH 101: | 15.00 |
| XL Kollidon: | 2.00 |
| L-HPC LH-11: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation V:

| | % |
|---|---|
| Fluoxetine TMMS*: | 28.69 |
| Pearlitol 400DC: | 36.31 |
| Emcompress: | 17.10 |
| Avicel PH 101: | 10.00 |
| XL Kollidon: | 2.00 |
| L-HPC LH-11: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation VI:

This formulation is based on an excipient mass containing a combination of low level of Avicel with Emcompress.

| | % |
|---|---|
| Fluoxetine TMMS*: | 28.69 |
| Pearlitol 400DC: | 43.81 |
| Emcompress: | 12.10 |
| Avicel PH 101: | 7.50 |
| XL Kollidon: | 2.00 |
| L-HPC LH-11: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation VII:

| | % |
|---|---|
| Fluoxetine TMMS*: | 28.69 |
| Pearlitol 400DC: | 48.41 |
| Emcompress: | 7.50 |
| Avice PH 101: | 7.50 |
| XL Kollidon: | 2.00 |
| L-HPC LH-11: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation VIII:

This formulation illustrates how the introduction of Clay (magnabrite) in tablet formulation according to the invention allows for covering the unpleasant and gritty taste of the microspheres and thereby improve the patient's ability to swallow a tablet based on this formulation.

| | % |
|---|---|
| Fluoxetine TMMS*: | 28.69 |
| Pearlitol 400DC: | 43.81 |
| Emcompress: | 12.10 |
| Avicel PH 101: | 6.50 |
| XL Kollidon: | 2.00 |
| L-HPC LH-11: | 2.00 |
| Magnabrite F: | 1.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation IX:

| | % |
|---|---|
| Fluoxetine TMMS*: | 28.69 |
| Pearlitol 400DC: | 43.81 |
| Emcompress: | 12.10 |
| Avicel PH 101: | 7.50 |
| XL Kollidon: | 2.00 |
| Magnabrite F: | 2.00 |
| Acesulfame K: | 0.20 |
| Magnasweet 100: | 0.20 |
| Tangerine Flavor: | 0.50 |
| Citric Acid anhydrous: | 1.50 |
| Syloid 244FP: | 0.50 |
| Pruv: | 1.00 |

### Formulation X:

| | % |
|---|---|
| Fluoxetine TMMS*: | 28.69 |
| Pearlitol 400DC: | 43.81 |
| Emcompress: | 12.10 |
| Avicel PH 101: | 7.50 |
| Magnabrite F: | 4.00 |
| AcesulfameK: | 0.20 |
| Magnasweet100: | 0.20 |
| Tangerine Flavor: | 0.50 |
| Citric Acid anhydrous: | 1.50 |
| Syloid 244FP: | 0.50 |
| Pruv: | 1.00 |

*Note: TMMS = Taste Masked Microspheres. Fluoxetine was used as a model drug, but these formulas cover the use of any coated or uncoated CEFORM ™ Microsphere. Reasonable variations, such as those which would occur to a skilled artisan, can be made herein without departing from the scope of the invention.

We claim:

1. A direct compression quick dissolve oral dosage form comprising:
(a) a drug-containing microparticle, and
(b) an excipient mass comprising:
(i) at least one of a directly compressible inorganic salt, a cellulose derivative, and a mixture thereof and
(ii) at least one directly compressible filler;
wherein said oral dosage form is a fast dissolving oral dosage form that dissolves in the mouth in less than about 40 seconds, has a friability of less than about 1%, and is manufactured by direct compression processing.

2. The oral dosage form of claim 1, wherein the drug-containing microparticle comprises at least one drug, and a combination of at least one solubilizer and at least one spheronization aid.

3. The oral dosage form of claim 1, wherein the excipient mass is comprised of about 50% directly compressible inorganic salt and about 50% cellulose derivative.

4. The oral dosage form of claim 1, wherein the excipient mass comprises at least one directly compressible inorganic salt selected from the group consisting of directly compressible dibasic calcium phosphate dihydrate, magnesium aluminum silicate NF, and mixtures thereof.

5. The oral dosage form of claim 1, wherein the excipient mass comprises a linear polyol.

6. The oral dosage form of claim 1, wherein the excipient mass comprises a directly compressible polyol.

7. The oral dosage form of claim 1, wherein the excipient mass further comprises mannitol; xylitol or a mixture thereof.

8. The oral dosage form of claim 1, wherein the excipient mass further comprises lactose, maltose, sucrose or a mixture thereof.

9. The oral dosage form of claim 1, wherein the drug-containing microparticles are liquiflash particles, and the drug-containing microparticles and the excipient mass are combined in proportions selected such that the drug remains within the liquiflash particles when the composition is compressed to obtain a dosage form having a hardness of from about 20 N to about 50 N.

10. The oral dosage form of claim 1, wherein the drug-containing microparticles particles are coated.

11. The oral dosage form of claim 1, wherein the drug-containing microparticles particles are coated with at least one taste-masking coating.

12. The oral dosage form of claim 10, wherein the coating contains at least one cellulosic polymer.

13. The oral dosage form of claim 10, wherein the coating comprises a polymethacrylate polymer.

14. The oral dosage form of claim 1, which dissolves in the mouth in less than about 30 seconds.

15. The oral dosage form of claim 1, wherein the excipient mass comprises a super disintegrant.

16. The oral dosage form of claim 1, wherein the excipient mass comprises from 0% to about 3% by weight of a super disintegrant.

17. The oral dosage form of claim 1, wherein said oral dosage form dissolves in the mouth in less than about 30 seconds and comprises from about 5% to about 45% by weight of drug-containing microparticles, and from about 25% to about 85% by weight of an excipient mass, wherein the excipient mass contains less than about 2.5% by weight of a super disintegrant.

18. The oral dosage form of claim 1, comprising from about 5% to about 20% by weight of microcrystalline cellulose.

19. The oral dosage form of claim 1, wherein the drug-containing microparticles comprise a drug selected from the group consisting of fluoxetine; paroxetine; zolpidem; tevenen; Cox-2 inhibitor; Ace inhibitor; a calcium channel blocker, and mixtures thereof.

20. The oral dosage form of claim 1, wherein the drug-containing microparticles comprise a drug selected from the group consisting of antitussives, antihistamines, decongestants, alkaloids, mineral supplements, laxatives, vitamins, antacids, ion exchange resins, anti-cholesterolemics, anti-lipid agents, antiarrhythmics, antipyretics, analgesics, appetite suppressants, expectorants, anti-anxiety agents, anti-ulcer agents, anti-inflammatory substances, coronary dilators, cerebral dilators, peripheral vasodilators, anti-infectives, psycho-tropics, antimanics, stimulants, gastrointestinal agents, sedatives, antidiarrheal preparations, anti-anginal drugs, vasodilators, anti-hypertensive drugs, vasoconstrictors, migraine treatments, antibiotics, tranquilizers, anti-psychotics, antitumor drugs, anticoagulants, antithrombotic drugs, hypnotics, anti-emetics, anti-nauseants, anti-convulsants, neuromuscular drugs, hyper- and hypoglycemic agents, thyroid and antithyroid preparations, diuretics, antispasmodics, uterine relaxants, mineral and nutritional additives, antiobesity drugs, anabolic drugs, erythropoietic drugs, antiasthmatics, cough suppressants, mucolytics, H₂-antagonists, anti-uricemic drugs and mixtures thereof.

21. The oral dosage form of claim 1, wherein the drug-containing microparticles comprise glyceryl stearate.

22. The oral dosage form of claim 1, wherein the drug-containing microparticle comprises hydroxypropylmethylcellulose.

23. The oral dosage form of claim 1, wherein the excipient mass comprises low substituted hydroxypropyl cellulose.

24. The oral dosage form of claim 1, wherein the excipient mass comprises microcrystalline cellulose.

25. The oral dosage form of claim 1, wherein the excipient mass comprises crospovidone.

26. The oral dosage form of claim 1, wherein the excipient mass comprises microcrystalline cellulose, crospovidone, and low substituted hydroxypropyl cellulose.

27. The oral dosage form of claim 1, wherein the excipient mass comprises mannitol.

28. The oral dosage form of claim 1, wherein the excipient mass comprises a sweetener.

29. The oral dosage form of claim 1, wherein the directly compressible filler comprises a directly compressible polyol.

30. The oral dosage form of claim 29, wherein the directly compressible polyol comprises at least one of mannitol, sorbitol, xylitol, or a mixture thereof.

31. The oral dosage form of claim 1, wherein the directly compressible filler comprises at least one of lactose, maltose, sucrose, dextrose, or a mixture thereof.
